(19) United States
(12) Patent Application Publication
(10) Pub. No.: US 2026/0053893 A1
(43) Pub. Date: Feb. 26, 2026

(54) ANAKINRA FOR THE TREATMENT OF POST ACUTE COVID SYNDROME

(71) Applicant: Swedish Orphan Biovitrum AB (publ), Stockholm (SE)

(72) Inventors: Fabrizio Dolfi, Basel (CH); Annik K.-Laflamme, Basel (CH); Evangelos Giamarellos-Bourboulis, Athens (GR)

(73) Assignee: Swedish Orphan Biovitrum AB (publ), Stockholm (SE)

(21) Appl. No.: 19/103,021

(22) PCT Filed: Aug. 11, 2023
(86) PCT No.: PCT/EP2023/072308

§ 371 Date: Feb. 11, 2025

(30) Foreign Application Priority Data
Aug. 12, 2022 (EP) 22190263.8

(51) Int. Cl. A61K 38/20; A61P 11/00; A61P 37/02; G01N 33/68

(52) U.S. Cl. CPC A61K 38/2006; A61P 11/00; A61P 37/02; G01N 33/6869; G01N 2333/522; G01N 2333/525; G01N 2333/5412; G01N 2333/545; G01N 2800/12; G01N 2800/32

(57) ABSTRACT

The present invention relates to the human Interleukin 1 Receptor antagonist anakinra for use in the treatment of Post-Acute COVID Syndrome (PACS), wherein said treatment comprises administration of a dose of at least approximately 100 mg of anakinra to a patient in need thereof. Also, related treatment methods and pharmaceutical compositions for said use are disclosed.

### FIELD OF THE INVENTION

The present invention relates to the human Interleukin 1 Receptor antagonist anakinra for use in the treatment of Post-Acute COVID Syndrome (PACS). Also, related treatment methods and pharmaceutical compositions for said use are disclosed.

### BACKGROUND

People with COVID-19 might have sustained post-infection sequelae, known as Post-Acute Covid Syndrome (PACS) (Soriano et al., Lancet Infect Dis 2022; 22: e102-e107). This is also described as long COVID or post COVID syndrome. The term PACS is used herein as an acronym to cover all entities described under the clinical condition of the constellation of symptoms which develops at least 60 to 90 days following COVID-19. A recent consensus definition by an international panel of 265 patients, clinicians, researchers, and WHO staff suggests that PACS occurs in individuals with a history of probable or confirmed SARS-CoV-2 infection, usually 3 months from the onset, with symptoms that last for at least 2 months and cannot be explained by an alternative diagnosis. Common symptoms include fatigue, shortness of breath, and cognitive dysfunction and generally have an impact on everyday functioning. Symptoms might be new onset after initial recovery from an acute COVID-19 episode or persist from the initial illness and they might also fluctuate or relapse over time (Soriano et al., 2022). The global incidence of PACS cannot be estimated but PACS might affect about 2.5% of Covid patients (https://www.nih.gov/news-events/nih-research-mattersAong-covid-symptoms-linked-inflammation).

Given the debilitating nature of the disease and its impact on daily activities, the high number of individuals affected, and the lack of treatments, there is a large and unmet medical need for new and improved therapies for treatment of PACS.

### SUMMARY OF THE INVENTION

It is an object of the present disclosure to provide new therapies for patients suffering from Post-Acute COVID Syndrome (PACS).

As used herein, the term “Post-Acute COVID Syndrome (PACS)” is to be interpreted as encompassing all entities described under the clinical condition of the constellation of symptoms which develops at least 60 to 90 days following COVID-19. A person of skill in the art will be familiar with alternative names and expressions, such as long COVID, long-haul COVID, post-acute sequelae of SARS CoV-2 infection (PASC), chronic COVID, long-term effects of COVID, Post-COVID Conditions (CDC) and Post Acute Coronavirus syndrome, (see, e.g., https://www.cdc.gov/coronavirus/2019-ncov/long-term-effects/index.html; Chippa, V. et al., StatPearls, NCBI Bookshelf (nih.gov); Nalbandian, A. et al., Nature Medicine 27, 601-615 (2021); Montani, D. et al., European Respiratory Review Covid-19 Review, 31 (2022); and Post-Acute COVID Syndrome (PACS): Definition, Impact and Management. A Report of the Multidisciplinary Collaborative Group for the Scientific Monitoring of COVID-19 (GCMSC), June 2021 (isglobal.org/en).

It is also an object of the present disclosure to provide a therapeutic agent which may be used alone or concomitantly with other agents for treatment of PACS, in particular to treat or alleviate one or more symptoms, such as fatigue, shortness of breath, and cognitive dysfunction, in subjects afflicted by PACS. Non-limiting examples of such other agents are respiratory drugs, cardiac drugs, anti-inflammatories and antidepressants.

According to a first aspect of the invention, this and other objects are achieved by anakinra for use in treatment of PACS in a subject, said treatment comprising administering to said subject a dose of anakinra.

In one embodiment, said dose is approximately 100 mg of anakinra.

As used herein, the term “approximately” is to be interpreted as ±5% in relation to a numeric value. Consequently, the term “approximately 100 mg” is to be understood as 95-105 mg. Thus, “approximately 100 mg anakinra” is to be understood as 95-105 mg anakinra.

In one embodiment, said dose is a daily dose.

In another aspect, the invention provides methods of treatment of PACS, and/or symptoms thereof, in subjects suffering from said PACS, said treatment comprising administering to said subject a dose of at least approximately 100 mg of anakinra.

In one embodiment, said dose is approximately 100 mg of anakinra. In one embodiment, said dose is a daily dose.

Suitably, said daily dose of anakinra is administered on the same time ±2 hours every day.

These, and other objects which are evident to the skilled person from the present disclosure, are met by the different aspects and embodiments of the invention as claimed in the appended claims and as generally disclosed herein.

The interleukin-1 family is a group of proteins involved in regulating immune and inflammatory responses. Anakinra is a recombinant form (17 kDa polypeptide) of a naturally occurring human interleukin-1 receptor antagonist (IL-1Ra) protein, functioning as a competitive inhibitor for receptor binding of IL-1. IL-1Ra has a balancing function with regard to other pro-inflammatory variants of IL-1. Anakinra blocks the binding of both IL-1α and IL-1β to the receptor. Anakinra also blocks the production of IL-6, a cytokine that is produced by PBMCs at significantly higher levels in patients recovering from COVID-19 pneumonia than comparators (see FIG. 2).

Anakinra is approved for treatment of rheumatoid arthritis, systemic juvenile idiopathic arthritis (sJIA) and adult-onset Still's disease and cryopyrin-associated periodic syndromes, at a dose of 100 mg once daily, or in weight-based doses up to 8 mg/kg/day, by subcutaneous (s.c.) injection.

However, anakinra has also been administered off-label in higher doses in critically ill patients suffering from macrophage activation syndrome (MAS)/sHLH and septic shock, including daily doses of up to 600 mg/day, with successful outcome (Grom A A et al., Nat Rev Rheumatol 2016, 12:259-68; Shakoory B et al., Crit Care Med 2016; 44:275-81; Kahn P J and Cron R Q, J Rheumatol 2013, 40:5; Fingerhutova et al., Frontiers in pediatrics, 7 Mar. 2022). Thus, in one embodiment the daily dose of anakinra may be approximately 100 mg, approximately 200 mg, approximately 300 mg, approximately 400 mg, approximately 500 mg, or approximately 600 mg or higher.

Intravenous (i.v.) administration of anakinra has been studied in clinical trials in healthy volunteers and in critically ill patients with sepsis and hyper-inflammation at variable i.v. doses up to 3500 mg/day over 72 hours: e.g. 2 mg/kg/hour, 20 mg/kg/day (<40 kg) and 916 mg/day (>40 kg), bolus of 100 mg followed by infusion of 2 mg/kg/hour, without safety concerns.

Analysis of patients that participated in a previous clinical trial, the SAVE-MORE randomized clinical trial (Kyriazopoulou et al., Nat Med 2021; 27: 1752-1760), has shown that survivors of COVID-19 pneumonia had persistent characteristics of a disequilibrium in hyper-/anti-inflammation and abnormal Th1 and Th2 responses irrespective of presence or absence of symptoms, when compared immunologically with healthy volunteers without any history of COVID-19 infection. Patients having received anakinra for their first COVID-19 episode were less probable to complain about PACS. All these results evidence a major role of the IL-1 pathway in the pathogenesis of PACS but also a major role of IL-1 inhibition in the prevention of PACS. These changes of cytokine production capacity of circulating monocytes and lymphocytes were associated with epigenetic modifications indicating that the changes were permanent and that the likelihood for self-resolution of PACS is limited.

The present invention was made in an attempt to alleviate one or more symptoms associated with PACS, such as fatigue, shortness of breath, cognitive dysfunction, hair loss, mood change, low back pain, arthralgias and sweats, and enable patients suffering from PACS to return to their normal daily activities as soon as possible.

The present inventors have identified that a dose of anakinra of at least approximately 100 mg, such as approximately 100 mg, may be useful in the treatment of PACS. In one embodiment said dose is a daily dose.

As used herein, the term “daily dose” refers to the total dose administered during one day to a patient.

As used herein, the term “day” is to be interpreted as a continuous 24-hour period.

As used herein, the term “administration occasion” refers to a limited time period wherein a drug is administered to a patient in need thereof. To clarify, the administration occasion encompasses the time period which is required for the administration of the entire dose of the drug which is to be administered at this time/occasion. For example, an administration occasion may be the time period required for one or several injections (e.g. subcutaneous injection), said one or several injections representing the complete dose to be administered at the specific time/occasion. Also, an administration of a dose which requires for example a 3-hour administration is to be regarded as one administration occasion. It is to be understood that the time period of an administration occasion is significantly shorter than the period in between two administration occasions.

The treatment with anakinra may be carried out or continued for at least 7 days, such as at least 14 days, such as at least 21 days, such as at least 28 days. Typically, the treatment is continued for 28 days (±2 days).

The dose of anakinra may be administered on day 1 to day 28 of a treatment period.

In some embodiments, a subject, such as a subject that has a recurrence of symptoms after a treatment period, may receive one or more additional treatment periods of, e.g., at least 7 days, such as at least 14 days, such as at least 21 days or such as at least 28 days. Thus, a subject that has a recurrence of symptoms after a treatment period of 28 days may, for example, receive treatment for an additional 7 days, 14 days, 21 days or 28 days, or more.

It will be appreciated that it is important that a patient experiences as little discomfort as possible during administration of a drug. Anakinra is typically administered as a subcutaneous injection to a patient.

Thus, in one embodiment, anakinra for use as disclosed herein is provided, wherein said administration is subcutaneous administration. The patient may be administered or may self-administer the drug at the hospital or in the comfort of his or her home environment. Thus, in preferred embodiments as disclosed herein, the administration of anakinra is subcutaneous injection, such as subcutaneous self-injection. In particular embodiments, the administration may be intravenous administration. Intravenous administration may, for example, be a useful option if a patient does not tolerate or is oversensitive to subcutaneous administration.

In some embodiments, the treatment may be combined with lung physiotherapy.

Suitably, the subject has a history of COVID-19 infection in the last 60 days or more, such as in the last 90 days or more. In some embodiments, said PACS develops at least 60 to 90 days following COVID-19.

In one embodiment, the subject to be treated experiences systemic symptoms without restriction in daily activities, restriction in daily activities with systemic symptoms or restriction in daily activities without systemic symptoms.

Typically, the subject to be treated experiences one or more, such as two or more, such as three or more, of the following: (i) needs help with daily activities but did not 30 need any help before getting sick by coronavirus; (ii) needs to stop and rest while doing a daily activity more often than before getting sick by coronavirus; (iii) finds it more difficult to do his/her daily job compared to before getting sick by coronavirus; (iv) needs more time to do manual work compared to before getting sick by coronavirus; (v) becomes more easily tired compared to before getting sick by coronavirus; (vi) feels more tired compared to before getting sick by coronavirus; (vii) has more of a problem having a shower or getting dressed compared to before getting sick by coronavirus; (viii) needs to use more effort to do daily activities compared to before getting sick by coronavirus; and (ix) finds it more difficult to climb stairs compared to before getting sick by coronavirus.

In particular, the subject to be treated may experience one or more of fatigue, hair loss, mood change, low back pain, arthralgias and sweats. In some instances, fatigue may be associated with exaggerated Th2 immune responses and an increase in serum concentrations of IL-1Ra, as demonstrated by the inventors in Example 1.

In Example 4, it was demonstrated by the inventors that different clusters of PACS could be identified. Those with fatigue, those with respiratory symptoms, those with systemic symptoms and those with other symptoms. The patients could be classified into one or more of these clusters, and the more classifications the worse PACS.

The treatment of PACS may be treatment of at least one of impaired lung function, impaired cardiac function and abnormal immune function. The inventors have found, as shown in Example 1, that patients recovering from COVID-19 pneumonia in need of hospitalization during acute infection present long-term post-infection immune dysregulation characterized by hyper-production of IL-1β and IL-6 from monocytes, and defects in Th2 and T17 responses.

In some instances, impaired lung function may be associated with aggravation of inflammation induced by monocytes reflected by high circulating calprotectin levels, as demonstrated by the inventors in Example 1.

In some instances, PACS may be characterized by increased cytokine production capacity. In particular, abnormal immune function may be associated with increased cytokine production, as shown by the inventors in Example 1, Example 3 and Example 4. As demonstrated in Example 4, the odds for IL-1β production greater than 400 pg/ml among PACS patients was 4.81. The respective odds for IL-6 production greater than 625 pg/ml was 11.75. Thus, cytokines such as IL-1α, IL-1β, IL-6, TNFα and/or IP-10 may be used as biomarkers or diagnostic tools to identify subjects with PACS. Moreover, the MMP-9, IL-17A and sCD163 may as demonstrated in Example 4 also be used as biomarkers for PACS. In one embodiment, IL-1α, IL-1β, IL-6, TNFα and/or IP-10 are used as diagnostic tools to identify subjects with PACS and/or subjects susceptible to developing PACS. In one particular embodiment, there is provided a diagnostic assay for identifying a subject that has PACS and/or is susceptible to developing PACS, comprising the steps of (i) measuring the levels of one or more cytokines selected from IL-1α, IL-1β, IL-6, TNFα and/or IP-10 in a sample isolated from the subject; and (ii) comparing said levels to those of a control sample, wherein a subject that has increased levels of said one or more cytokines compared to the control sample is identified as having PACS and/or being susceptible to developing PACS. Suitably, said one or more cytokines is IL-1β or IL-6.

In some embodiments, PACS may be characterized by increased serum levels of interferon-gamma-induced protein-10 (IP-10). In particular, increased levels of IP-10 may be associated with increased potential for the production of IL-1β by monocytes and decreased production of IL-1ra, as shown by the inventors in Example 3. Being the soluble antagonist of the receptor IL-1R1 of IL-1, IL-1ra acts as an anti-inflammatory mediator. In particular, serum levels of IP-10 of more than 250 pg/ml may indicate increased biosynthesis of IL-1β and decreased biosynthesis of IL-1ra. The IP-10 serum level can be used as an index of the pro-inflammatory state of the patients, and potentially as a biomarker or diagnostic tool to identify subjects with PACS and/or hyperinflammation. Thus, in some instances, the subject to be treated has an IP-10 serum level of at least 250 pg/ml.

In some embodiments, the subject to be treated may have lungs with reduced ability to transfer oxygen to blood (i.e. have low diffusion capacity), an abnormal chest, and/or abnormal immune function.

In the present context, the following may be signs of impaired lung function: reduced lung diffusion or carbon monoxide capacity (DLCO), such as a DLCOcor <76%, and a total lung capacity (TLC) and/or forced vital capacity (FVC) lower than 80% of predicted.

In the present context, the following may be signs of an abnormal chest at least a total radiology score in high-resolution chest tomography (HRCT) of more than 20 or walking of a distance less than 500 m in the 6-minute walk test. A person of skill in the art will be familiar with the term HRCT and how it is used to test for, diagnose and monitor abnormalities and diseases of the lung tissue and the airways in a subject, in light of the list of abbreviations below, the information provided in the Examples and technical knowledge.

In some instances, impaired cardiac function may be associated with a decrease of LV ejection fraction (LVEF), wherein the heart's ability to pump oxygen-rich blood out to the body is reduced. A person of skill in the art will be familiar with the term LVEF, how it is measured and how it helps determine the severity of dysfunction on the left side of the heart, in light of the list of abbreviations below, the information provided in the Examples and technical knowledge.

In the present context, the following may be signs of abnormal immune function: excessive IL-1β levels of more than 2500 pg/ml after stimulation of peripheral blood mononuclear cells (PBMCs) with lipopolysaccharide (LPS). The skilled person will be familiar with the term abnormal immune function and how a subject is tested for abnormal immune function in light of the list of abbreviations below, the information provided in the Examples and technical knowledge.

Suitably, said treatment results in at least one of (i) at least 20% improvement of restrictive lung disease, such as an increase of at least one of DLCOcor, TLC, FVC and FEV1/FVC from baseline; (ii) no need for hospitalization; and (iii) no increase of the degree of lung fibrosis score in lung HRCT. Suitably, said treatment results in at least two of (i) to (iii).

Suitably, said treatment results in at least one of (i) at least 20% decrease of the total radiology score in lung HRCT or improved exercise capacity in the 6-minute walk test; (ii) no need for hospitalization; and (iii) no increase of the degree of lung fibrosis score in lung HRCT. Suitably, said treatment results in at least two of (i) to (iii).

Suitably, said treatment results in reduced cytokine production capacity of LPS-stimulated PBMCs; at least 10% decrease of the pulmonary artery pressure; and/or at least 10% increase of LV ejection fraction.

In another aspect of the present disclosure, there is provided a pharmaceutical composition comprising anakinra for use as described herein and at least one pharmaceutically acceptable excipient, diluent and/or carrier.

The term “pharmaceutical composition” is to be used in its widest sense, encompassing all pharmaceutically applicable compositions containing at least one active substance, and optional carriers, adjuvants, constituents etc. The term “pharmaceutical composition” also encompasses a composition comprising the active substance in the form of derivate or a prodrug, such as a pharmaceutically acceptable salt, sulphate and/or ester of said active substance. The active substance may be anakinra. The manufacture of pharmaceutical compositions for different routes of administration, such as subcutaneous administration, falls within the capabilities of a person skilled in galenical chemistry.

In one embodiment, the pharmaceutical composition is Kineret®, a formulation of anakinra at pH 6.5 suitable for injection, such as subcutaneous injection, that contains sodium citrate, sodium chloride, disodium EDTA, polysorbate 80 and water.

It is also contemplated that said treatment may be useful not only as a therapeutic treatment of PACS but also a preventive treatment of PACS. Thus, in one embodiment, said treatment is preventive treatment of PACS. Such a treatment may be particularly suitable for subjects that are susceptible to developing PACS, for example patients recovering from COVID-19 pneumonia in need of hospitalization during the acute phase of the infection that have abnormally high IL-1 and/or IL-6 levels.

Typically, the subject is a human subject.

In particular, it is noted that, as used in this specification and the appended claims, the singular forms “a”, “an”, and “the” also include plural referents unless the context clearly dictates otherwise.

While the invention has been described with reference to various exemplary aspects and embodiments, it will be understood by those skilled in the art that various changes may be made and equivalents may be substituted for elements thereof without departing from the scope of the invention. In addition, many modifications may be made to adapt a particular situation or molecule to the teachings of the invention without departing from the essential scope thereof. Therefore, it is intended that the invention not be limited to any particular embodiment contemplated, but that the invention will include all embodiments falling within the scope of the appended claims.

### BRIEF DESCRIPTION OF THE FIGURES

FIG. 1. Study flow chart. The discovery set (discovery cohort) was composed of 46 patients sampled at 100 days from hospital discharge and 25 matched comparators. The validation set (validation cohort) composed of 457 patients 120 days post hospital discharge who participated in the previous SAVE-MORE (Kyriazopoulou et al., supra) randomized clinical trial. Patients of the validation cohort were treated during acute illness either with placebo or anakinra. Those who were allocated to placebo treatment were further sampled for immune function and subject to lung function tests. Twenty-one matched comparators were also studied. Participants in the SAVE-MORE trial were also contacted by phone visits one year after hospital discharge.

FIG. 2. Activation of monocytes/macrophages after COVID-19 infection. a) Production of interleukin (IL)-1β and b) IL-6 from peripheral mononuclear blood cells (PBMCs) after stimulation with lipopolysaccharide (LPS) from the discovery cohort (September-October 2020) and comparators. P values by the non-parametric Mann-Whitney comparison between patients and comparators are provided. Similar comparisons between patients and comparators, c) and d), during the study period June-August 2021. P values by non-parametric Mann-Whitney comparison between patients and controls are provided.

FIG. 3. Grouping of symptoms in patients with post-acute Covid syndrome (PACS). a) Heatmap of symptoms in the validation cohort. The non-parametric Spearman co-efficient of correlation (rₛ) and the respective p-values are provided. b) Main symptoms of the ST-IT questionnaire for LCS in the validation cohort.

FIG. 4. Impact of anakinra treatment in the development of PACS. Classification of PACS into Groups I to IV according to the 16-symptom questionnaire among survivors of the SAVE-MORE trial 120 days after hospital discharge. The comparison between patients allocated to treatment with placebo and treatment with anakinra is done by ordinal regression analysis. The two assumptions of the ordinal regression Goodness-of-fit and proportional odds are also provided. The two assumptions of the ordinal regression were not significant showing that the likelihood that the difference between the group of placebo and anakinra is random is limited. (Goodness-of-fit test (Pearson's chi-square test): P: 0.751; Assumption of proportional odds (test of parallel lines): P: 0.742) FIG. 5. Immune dysregulation following COVID-19 infection. a) Comparison of T-cell derived cytokines between patients of the validation cohort and respective comparators. Measured interferon (IFN)-gamma and tumor necrosis factor-alpha (TNFα) are indicators of Th1 responses; interleukin (IL)-10 of Th2 responses and IL-17 of Th17 response. The p-values of comparisons between patients and comparators are provided. b) Comparison of the monocyte production capacity of IL-1β and IL-6 depending on the time since onset of COVID-19 pneumonia. The p-values of comparisons are provided.

FIG. 6. Immune dysregulation and symptoms of PACS. a) Concentrations of soluble interleukin (IL)-1Ra antagonist among patients with and without fatigue. b) Concentrations of IL-1α released from circulating monocytes following stimulation with bacterial lipopolysaccharide (LPS) among patients with and without fatigue. c) Concentrations of IL-10 released from PBMCs following stimulation with heat-killed Candida albicans among patients with and without fatigue. d) Concentrations of S100A8/A9 (calprotectin) among patients after COVID-19 pneumonia with (Yes) and without (No) exertional dyspnea. e) and f) Activation of monocytes/macrophages as manifested by the release of IL-1β and IL-6 during incubation of mononuclear cells with lipopolysaccharide in patients after COVID-19 pneumonia who present with normal or abnormal lung function tests (LFT). P values by non-parametric Mann-Whitney comparisons are provided.

FIG. 7. General scheme of the SAVE-LONG RCT (this Figure belongs to Example 2 below).

FIG. 8. Comparative production of a) interleukin (IL)-1β and b) IL-6 by circulating monocytes of patients of the validation cohort following stimulation with bacterial lipopolysaccharide. Participants were classified into four groups of long-COVID syndrome (Group I: systemic symptoms without restriction in daily activities; Group II: restriction in daily activities with systemic symptoms; Group III: restriction in daily activities without systemic symptoms; and Group IV: asymptomatic). The p-value of non-parametric comparison between patients classified into Groups I to III, into Group IV and comparators are provided.

FIG. 9. Association between groups of post-acute Covid symptoms and patient reported outcomes defined by A)-B) the Short Form (SF)-36 Health Survey and C) the Symptom Tool-Impact Tool (ST-IT) questionnaires. The cumulative responses to the six main elements of the SF-36 health survey are provided according to the four groups of symptoms. The number of answers of the ST-IT questionnaires for which patients provided positive answers are also provided. P values represent comparisons with Group IV after ANOVA and Bonferroni corrections.

FIG. 10. Impact of anakinra treatment in the development of PACS. Comparison of the main elements of the Short Form-36 Health Survey. The comparison between patients allocated to treatment with placebo and treatment with anakinra is done by the Student's t-test. Results are adjusted so that the scale 100 indicates better performance towards the values of 100.

FIG. 11. Impact of anakinra treatment in the development of PACS 360 days after hospital discharge. Classification of PACS into Groups I to IV according to the 16-symptom questionnaire among survivors of the SAVE-MORE trial 360 days after hospital discharge. The comparison between patients allocated to treatment with placebo and treatment with anakinra is done by ordinal regression analysis.

FIG. 12. Circulating levels of biomarkers between participants in the validation cohort and respective comparators. a) C-reactive protein (CRP), b) ferritin, c) interleukin (IL)-6, d) interferon (IFN)-gamma, e) IL-RA antagonist, f) IL-18, g) IL-33r, h) troponin I, and i) NT-proBNP. P values by non-parametric Mann-Whitney comparisons are provided.

FIG. 13. Impact of anakinra treatment in the development of post-acute Covid syndrome (PACS). The percentage of survivors of the SAVE-MORE trial 120 days after hospital discharge is classified into asymptomatic and into presenting one or more phenotype clusters of PACS. The comparison between patients allocated to treatment with placebo and treatment with anakinra is done by ordinal regression analysis. The two assumptions of the ordinal regression (Goodness-of-fit and proportional odds) are also provided. The two assumptions of the ordinal regression were not significant showing that the likelihood for the difference between the groups to be random is limited. Abbreviations: Cl, confidence intervals; OR, odds ratio; SoC, standard-of-care.

FIG. 14. Immune function by PACS phenotype. A) Patients with the fatigue cluster of symptoms have decreased production of the production of TNF (tumor necrosis factor) by monocytes and decreased circulating levels of the metallo-proteinsae-9 (MMP-9) which is an index of neutrophil activation. B) Patients with the respiratory cluster of symptoms present with increase in net function for the production of IL-1β and IL-1α by monocytes and by increased circulating levels of IL-17A and sCD163 (index of tissue macrophage activation).

FIG. 15. IP-10 as an index of PACS immune dysregulation. A) ROC curve of IP-10 for the discrimination between patients and comparators. B) Performance of IP-10 more than 250 pg/ml for the diagnosis of PACS. C) Comparative production of IL-1β by blood monocytes in accordance to the concentrations of IP-10. D) Comparative production of IL-1ra by blood monocytes in accordance to the concentrations of IP-10. Abbreviations AUC: area under the curve, Cl: confidence intervals, IL: interleukin, NPV: negative predictive value; PPV: positive predictive value.

FIG. 16. General scheme of the PRECISION clinical study (Example 3) FIG. 17. Study flow chart for Example 4. The discovery set was composed of 46 patients sampled at 100 days from hospital discharge and 25 matched comparators. The validation set was composed of two cohorts. Cohort 1 included 464 patients who participated in the SAVE-MORE randomized clinical trial and who were evaluated for their symptoms 120 days post hospital discharge. Patients of the validation cohort 1 were treated during acute illness either with placebo or anakinra. Several patients were further sampled for immune function and subject to lung function tests. Fifty-eight matched comparators were also studied. Participants in the SAVE-MORE trial were also contacted by phone visits one year after hospital discharge. Validation cohort 2 included 130 patients who were admitted in an out-patient infectious diseases unit for follow-up and who were evaluated for their symptoms 120 days post hospital discharge. Abbreviations: DLCO: carbon monoxide lung diffusion; FEV1: forced expiratory volume in one second; FVC: forced vital capacity; IL: interleukin; PACS: post-acute COVID syndrome; PBMCs, peripheral mononuclear blood cells; SF-36: Short Form 36 health survey; SoC: standard-of-care; ST-IT, Symptom Tool-Impact Tool for Long Covid Syndrome; TLC: total lung capacity.

FIG. 18. Activation of PBMCs post COVID-19 pneumonia. A, B) Production of IL-1β and IL-6 from PBMCs of comparators and patients of the two validation cohorts after stimulation with LPS. Validation cohort 1 is coming from patients enrolled in the SAVE-MORE trial at a median time of 120 days post hospital discharge i.e. June-August 2021. Patients of this cohort are split into those who received treatment in the SAVE-MORE trial with standard-of-care (SoC) and placebo or with SoC and anakinra. Validation cohort 2 is coming from patients who were admitted with COVID-19 pneumonia one year later and who were enrolled at a median time of 120 days post hospital discharge i.e. June-August 2022. In every panel, lines represent the median of distribution and 95% confidence intervals. P values by the non-parametric Mann-Whitney test are provided: *p<0.05; *p<0.01; ***p<0.001; ****p<0.0001. Abbreviations IL, interleukin; LPS, lipopolysaccharide; n, number of patients.

FIG. 19. Phenotype clusters in patients with post-acute Covid syndrome (PACS). A) Main clusters of symptoms among patients of both validation cohorts using the 16-element questionnaire. The fatigue cluster is composed by positive replies to at least two of the questions of difficulty in daily activity, easy tiring, difficulty at manual work, difficulty at climbing stairs, need to rest, need of assistance for bathing and dressing and feeling of fatigue. The respiratory cluster is composed by positive replies to at least one of need of oxygen and difficulty at work. The systemic symptoms cluster is composed by positive replies to at least one of production of sputum and presence of fever or cough. The cluster of others is composed by positive replies to at least one of difficulty in concentration and alteration of taste or smell. B) The fatigue cluster is validated by the respective domains of the SF-36 questionnaire. Patients classified to the fatigue cluster (Yes) and patients classified to other clusters (No) are compared for the self-points to the five questions of SF-36 characterizing fatigue. Positive symptoms deliver less points. The p-values of comparison by the Student's t-test are provided. C) The respiratory cluster is validated by the respective domain of the SF-36 questionnaire. Patients classified to the respiratory cluster (Yes) and patients classified to other clusters (No) are compared for the self-points to the questions of SF-36 characterizing respiratory dysfunction. Positive symptoms deliver less points. The p-value of comparison by the Student's t-test is provided. D) The cluster of systemic symptoms is validated by the respective domain of the SF-36 questionnaire. Patients classified to the systemic cluster (Yes) and patients classified to other clusters (No) are compared for the self-points to the questions of SF-36 characterizing the systemic symptoms. Positive symptoms deliver less points. The p-value of comparison by the Student's t-test is provided. E) The fatigue cluster is validated by the respective questions of the ST-IT questionnaire. Patients classified to the fatigue cluster (Yes) and patients classified to other clusters (No) are compared by the number of their positive replies to the fatigue domain of the ST-IT questionnaire. The p-values of comparisons by the Student's t-test are provided. F) The respiratory cluster is validated by the positive findings of lung function tests (LFTs). Patients classified to the respiratory cluster (Yes) and patients classified to other clusters (No) are compared by the rate of positive findings on spirometry (i.e. corrected DLCO less than 70% and/or functional vital capacity less than 80%). The p-value of comparison by the Fisher exact test is provided. Abbreviations: Cl, confidence intervals; SF-36, short form 36 health survey; ST-IT, Symptom Tool-Impact Tool for Long Covid Syndrome.

FIG. 20. Modulation of circulating mediators in post-COVID pneumonia. Concentrations of serum mediators in healthy comparators and patients post-Covid are shown. In all panels, lines represent the median of distribution and 95% confidence intervals. The p-values of comparisons by the Mann-Whitney U test are provided: ns, non-significant; *<0.05; **<0.01;***<0.001; **<0.0001. Abbreviations CRP: C-reactive protein; FGF: fibroblast growth factor; IFN, interferon; IL, interleukin; IP, interferon-gamma induced protein; MMP, metalloproteinase; n, number of patients; PDGF, platelet-derived growth factor.

FIG. 21. Cytokine stimulation in post-COVID-19 pneumonia. A) Cytokine responses following stimulation of peripheral blood mononuclear cells (PBMCs) of comparators and of post-COVID patients. Stimulation is performed with bacterial lipopolysaccharide (LPS) targeting the monocyte fraction of PBMCs or heat-killed Candida albicans (HKCA) targeting the lymphocyte fraction of PBMCs. Patients of the validation cohort 1 treated with placebo for the acute COVID-19 and patients of the validation cohort 2 are presented together in this analysis and they are indicated as SoC. B) IL-1β and IL-6 responses following stimulation from PBMCs of survivors from COVID pneumonia collected at follow-up time points. Stimulation was performed with LPS targeting the monocyte fraction of PBMCs. In all panels, lines represent the median of distribution and 95% confidence intervals. The p-values of respective comparisons are provided: ns, non-significant; *<0.05; *<0.01; **<0.001; ****<0.0001. Abbreviations IFN, interferon; IL, interleukin; n, number of patients; SoC, standard-of-care; TNF, tumor necrosis factor.

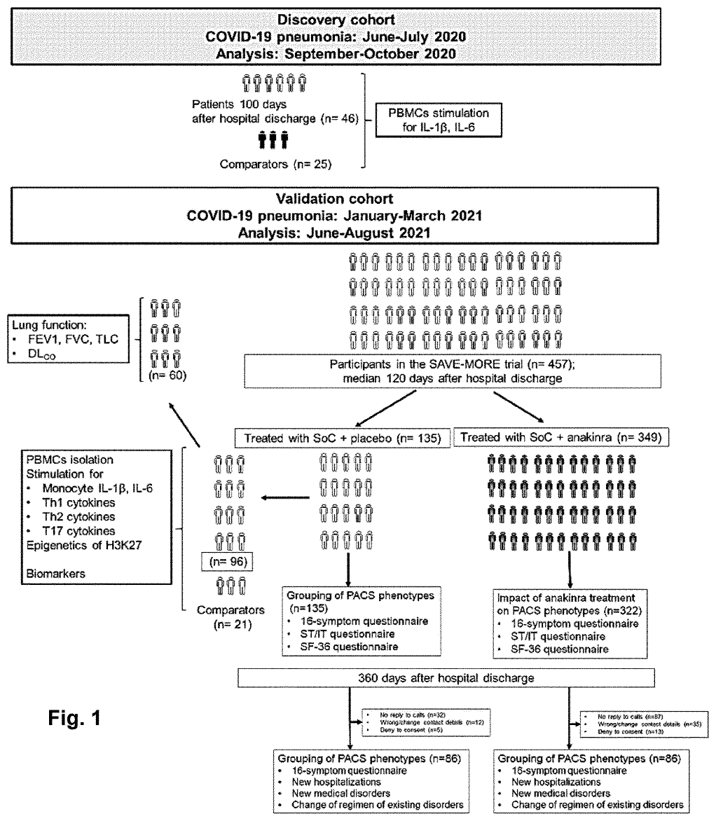

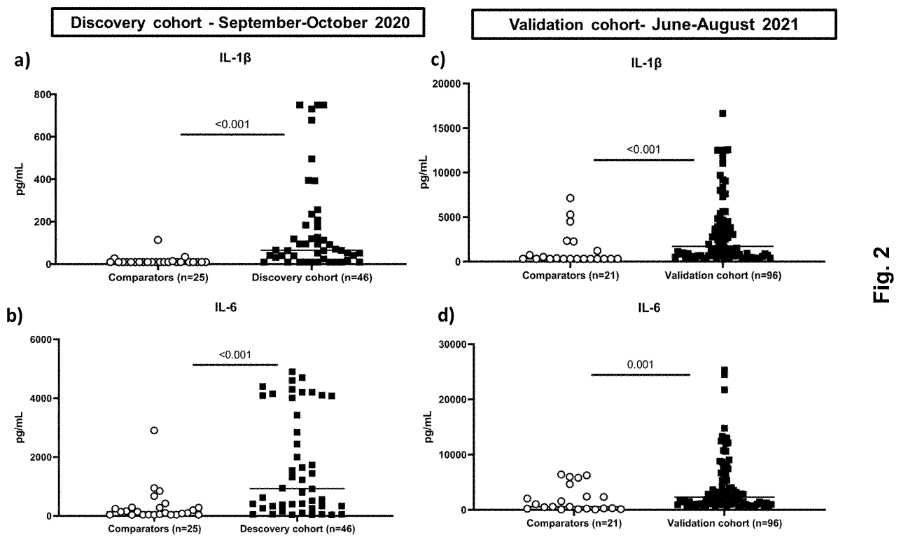

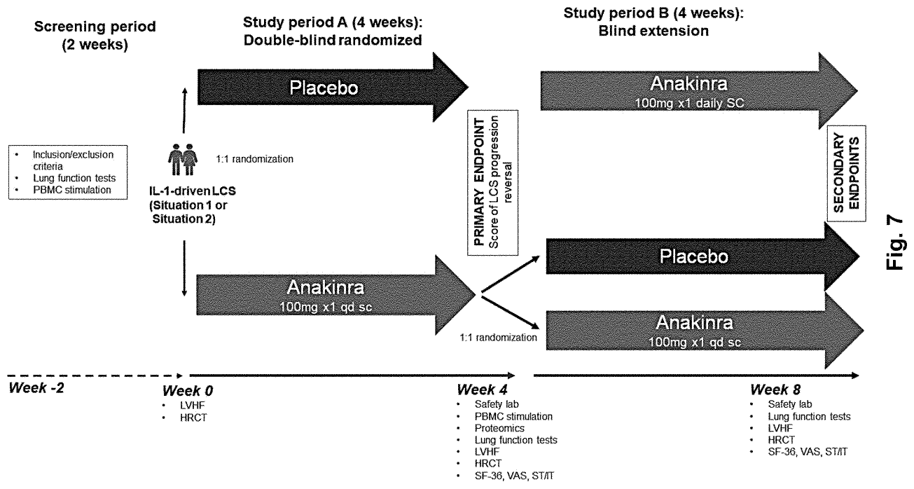

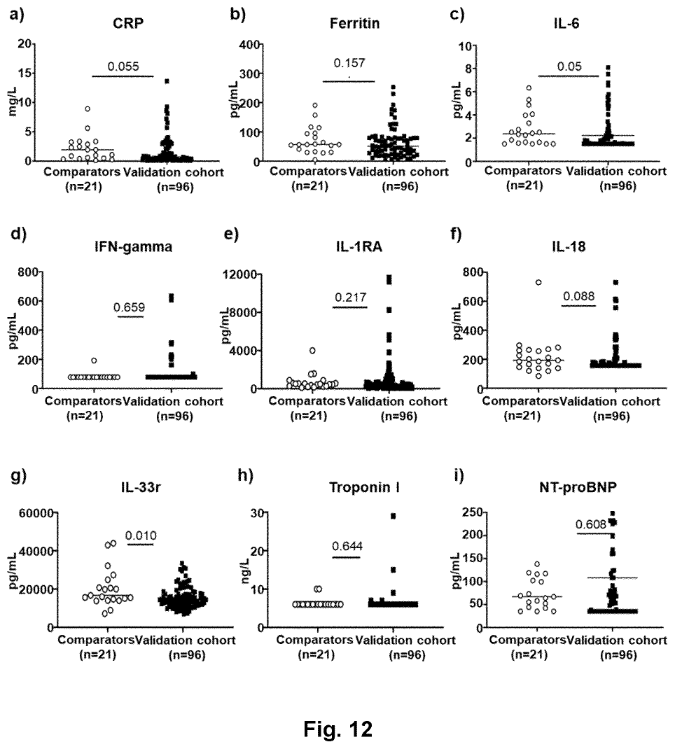

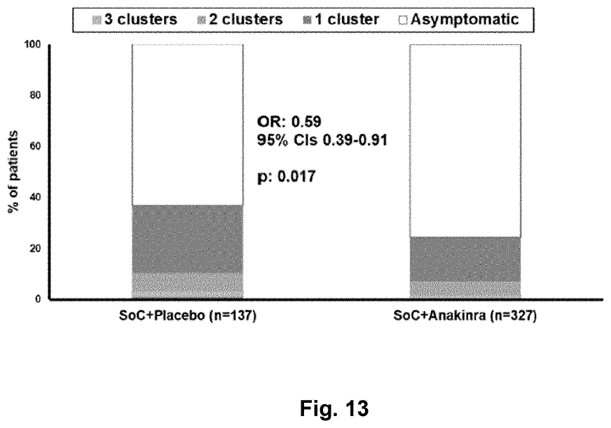

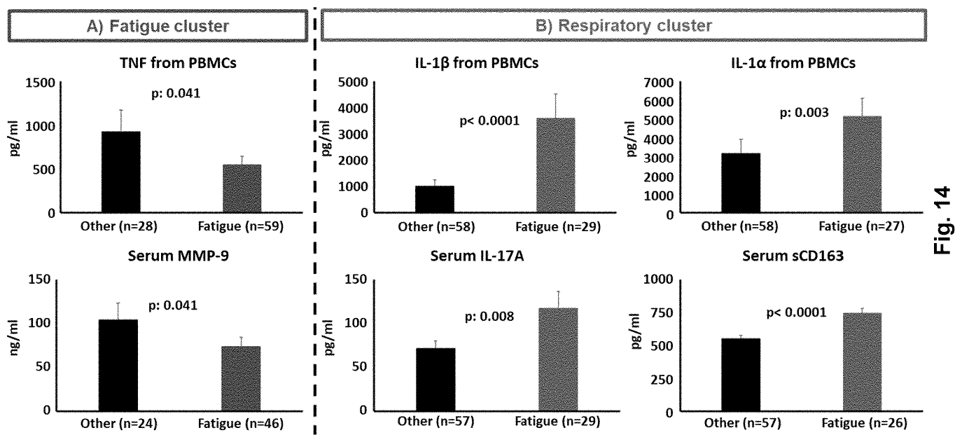

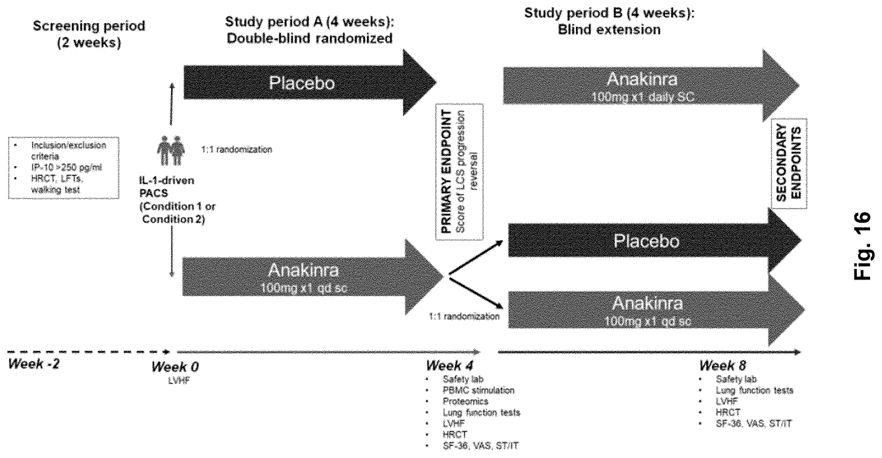

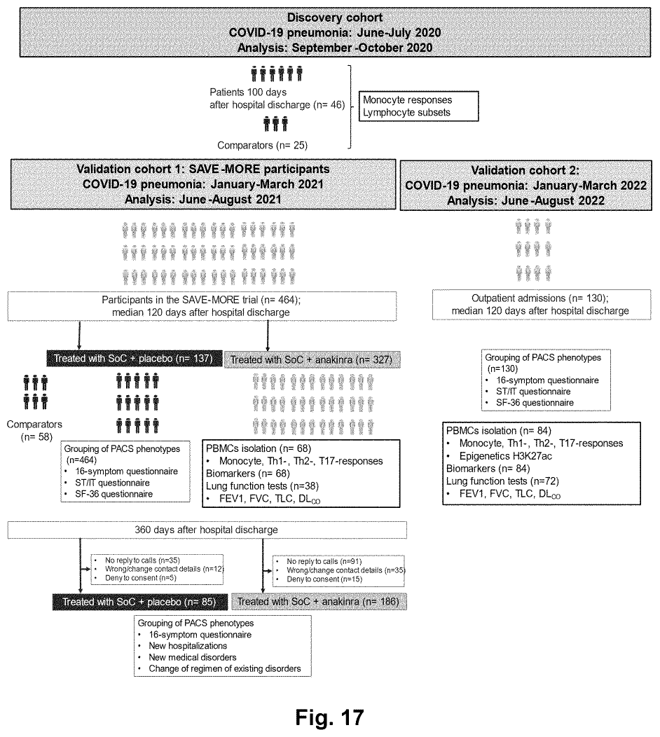

FIGS. 1-6 and 8-12 belong to Example 1 below, while FIG. 7 belongs to Example 2 below, FIGS. 14-16 to Example 3 below, and FIGS. 13,17-21 to Example 4 below.

### DETAILED DESCRIPTION

Novel coronavirus-2 (SARS-CoV-2) infection, also known as COVID-19 disease, is characterized by great heterogeneity in clinical presentation ranging from asymptomatic disease to severe pneumonia with respiratory distress and need for respiratory support and mechanical ventilation (Grasselli, G. et al., JAMA Intern Med 180, 1345-1355, 2020). Inappropriate activation of innate and adaptive immune response, e.g. hyperinflammation mediated by IL-1 and IL-6 (RECOVERY Collaborative Group, Lancet 397, 1637-1645, 2021), appears to play a central role in the pathogenesis of COVID-19 pneumonia.

People with COVID-19 might have sustained post-infection sequelae, known as Post-Acute Covid Syndrome (PACS) (Soriano J B et al., Lancet Infect Dis 2022; 22: e102-e107). This syndrome seems to occur in individuals with a history of probable or confirmed SARS-CoV-2 infection, usually 3 months from the onset, with symptoms that last for at least 2 months and cannot be explained by an alternative diagnosis. Common symptoms include fatigue, shortness of breath, and cognitive dysfunction and generally have an impact on everyday functioning.

### EXAMPLES

### Summary

The present Examples describe 1) a background clinical study of the immune dysregulation of IL-1 and IL-6 production in patients recovering from COVID-19 and their involvement in the pathogenesis of PACS. This study, which, inter alia, indicated that anti-IL-1 therapy with anakinra during the acute phase of the disease has the potential to significantly decrease the incidence of PACS, have prompted the investigators to undertake 2) a clinical study (SAVE-LONG) that aims to assess the efficacy and safety of the hIL-1R antagonist anakinra for treating PACS, 3) a clinical study (PRECISION) that aims to evaluate the efficacy and safety of anakinra in patients with Post-Acute COVID Syndrome (PACS) of the pro-inflammatory respiratory type, and 4) a study of long-term immune and epigenetic dysregulation following covid-19.

Anakinra is a recombinant, non-glycosylated form of the human interleukin 1 receptor antagonist (IL-1Ra). The amino acid sequence of anakinra is identical to the naturally occurring protein except for the addition of an N-terminal methionine residue.

Anakinra is a 153-amino acid protein with an approximate molecular weight of 17.3 kDa. Anakinra is produced by recombinant DNA technology in an E. coli expression system. Therapeutically, anakinra neutralizes the biological activity of IL-1 (IL-1α and IL-1β) by competitively inhibiting its binding to the IL-1R-I.

Components in a protein drug formulation of anakinra (“the Study drug”) may include buffering agents, tonicity agents, antioxidants, stabilizers, surfactants, bulking agents, chelating agents and preservatives. The present studies use Kineret®, a formulation of anakinra at pH 6.5 suitable for injection, such as subcutaneous injection, that contains sodium citrate, sodium chloride, disodium EDTA, polysorbate 80 and water.

### Abbreviations and Definition of Terms Used Herein

| Abbreviation | Definition |
|---|---|
| AE: | Adverse event |
| ALT: | Alanine aminotransferase |
| AST: | Aspartate aminotransferase |
| aPTT: | Activated partial thromboplastin time |
| CCI: | Charlsons' comorbidity index |
| CI: | Confidence interval |
| COVID-19: | Coronavirus 2019 disease |
| CRA: | Clinical research associate |
| CRP: | C-reactive protein |
| DLCO: | Lung diffusion of carbon monoxide capacity |
| eCRF: | Electronic case report form |
| ED: | Emergency department |
| EDTA: | Ethylene-diamine-tetraacetic acid |
| FEV1: | Forced expiratory volume in one second |
| FVC: | Forced vital capacity |
| γGT: | Gamma-glutamyl transpeptidase |
| HRCT: | High-resolution chest computed tomography |
| IFN: | Interferon |
| IL: | Interleukin |
| INR: | International normalized ratio |
| IT: | Impact tool for Long Covid |
| ITT: | Intention-to-treat |
| IV: | Intravenous |
| LCS: | Long-COVID syndrome |
| LOCF: | Last observation carried forward |
| LPS: | Lipopolysaccharide |
| MCH: | Mean corpuscular hemoglobin |
| MCHC: | Mean corpuscular hemoglobin concentration |
| MCV: | Mean corpuscular volume |
| MV: | Mechanical ventilation |
| NA: | Not applicable |
| NPV: | Negative predictive value |
| OR: | Odds ratio |
| PACS: | Post-acute Covid syndrome |
| PBMC: | Peripheral blood mononuclear cells |
| PCS: | Post covid syndrome |
| Q: | Quartile |
| qd: | Once daily |
| RCT: | Randomized clinical trial |
| RT-PCR: | Real-time polymerase chain reaction |
| SAE: | Serious adverse event |
| SAP: | Serum alkaline phosphatase |
| SARS-CoV-2: | Severe acute respiratory syndrome coronavirus-2 |
| SD: | Standard deviation |
| sc: | Subcutaneously |
| SF-36: | Short Form 36 health survey |
| SIV: | Site initiation visit |
| SoC: | Standard-of-Care |
| ST: | Symptom tool for Long Covid |
| sTEAE: | Severe treatment-emergent adverse event |
| TEAE: | Treatment-emergent adverse event |
| TLC: | Total lung capacity |
| VAS: | Visual analogue scale |
| WHO | World Health Organization |
| WHO CPS: | WHO clinical progression scale |

### Example 1

### Clinical Study of Lona Term Immune and Epigenetic Dysregulation Following COVID-19

### Methods

### Trial Oversight

This study was prospective, observational and it was conducted in two outpatient post-covid units in Greece. The protocol was approved by the Ethics Committee of the participating hospitals (Attikon University Hospital 1-11/1/2022 and General Hospital of Piraeus Tzaneio 16-14/5/2021). The study was sponsored and funded by the Hellenic Institute for the Study of Sepsis (HISS), which was also responsible for the design, conduct, analysis and interpretation of data and the decision to publish. The laboratory of Immunology of Infectious Diseases of the 4^{th} Department of Internal Medicine at ATTIKON University General Hospital served as the central laboratory.

### Patients

Inclusion criteria were: a) adults aged 18 years or older; b) history of hospitalization for COVID-19 pneumonia the last 3 to 6 months; and c) negative molecular test for SARS-CoV-2. Exclusion criteria were: a) any stage IV malignancy; b) any primary immunodeficiency; c) neutropenia with fewer than 1,500 neutrophils per mm³; d) oral or intravenous intake of corticosteroids at a daily dose greater than or equal to 0.4 mg kg⁻¹ of prednisone or equivalent for a period longer than 15 consecutive days the last 3 months; and e) any chronic anti-cytokine biological treatment. Patients were divided in two cohorts (FIG. 1): a) one discovery cohort of patients hospitalized in June-July 2020 and included in the current study in September-October 2020; and b) one validation cohort consisting of patients, participants in the SAVE-MORE randomized clinical trial, hospitalized between January and March 2021 and included in current study between June and August 2021 (Kyriazopoulou, et al., supra). All patients or their legal representatives provided written informed consent before enrolment.

### Study Procedures

From patients of both cohorts, 20 mL of blood sample was drawn from a peripheral forearm vein under aseptic conditions. The samples were collected into a) tubes with ethylenediaminetetraacetic acid (EDTA) and transferred within one hour to the central laboratory for isolation and culture of peripheral blood mononuclear cells (PBMCs) and b) in sterile pyrogen-free tubes for serum collection. For all patients, the following information was collected: date of COVID-19 infection, severity of disease, demographics (age, gender) and Charison's comorbidity index (CCI). Similar blood sampling was repeated in some patients of the validation cohort 12 months after hospitalization for COVID-19 pneumonia and in comparators without known history of COVID-19 infection, irrespective from vaccination status and matched for age, sex and CCI.

Patients of the validation cohort (SAVE-MORE participants) were requested to provide answers to three different questionnaires; the first contained 16 questions to be answered as either Yes or No and developed by the researchers as a tool of quality of life used in the SAVE-MORE trial (Kyriazopoulou, et al., supra); the second was the ST-IT questionnaire which is internationally proposed for follow up of COVID-19 patients (Tran, V. T. et al., Clin Infect Dis 74, 278-287, 2022); and the third was the SF-36 health survey. The 16 questions of the first questionnaire concerned the presence one or more of the following: long-term home oxygen therapy, difficulty in concentration, need for assistance in everyday activities, need to rest during an activity, difficulty in doing a daily activity, dyspnea, fever/cough, sputum, difficulty in manual work, difficulty in climbing stairs, easy tiredness, feeling of fatigue, difficulty to bathe or dress, altered taste and smell. Patients with history of COVID-19 infection were classified as LCS if they answered positive at least one question of the 16-symptom questionnaire and if the symptom could not be explained by an alternative diagnosis. To exclude an alternative diagnosis, patients underwent thorough clinical, laboratory and radiology evaluation by their attending physicians.

In patients of the validation cohort lung function tests were performed including spirometry, plethysmography and diffusing capacity for carbon monoxide (Elite Series Plethysmograph, Medical Graphics, Minnesota, USA).

### PBMC Preparation and Cytokine Measurements

PBMCs were isolated using gradient centrifugation over Ficoll-Hypaque density gradient (Biochrom, Berlin, Germany) after 1400 g under 4° C. centrifugation over 25 minutes. After three washings in ice-cold PBS (phosphate buffered saline, pH: 7.2, Biowest, France), PBMCs were counted using a Neubauer plate with trypan blue exclusion of dead cells. PBMCs were incubated in 1 mL RPMI 1640 (Biochrom, Berlin, Germany) enriched with 100 U/ml penicillin G and 0.1 mg/mL streptomycin and distributed on 96-well plates at final volume 200 μL and density 5×10°/ml. Cells were incubated at 37° C. and 5% CO₂ for 24 hours without/with 10 ng/ml lipopolysaccharide (LPS) of Escherichia coli O55:B5. Cells were also incubated in same circumstances for five days with 10% fetal bovine serum without/with 5×10⁶ cfu/mL heat-killed Candida albicans (HKCA). After incubation the supernatants were collected and the following were measured in duplicate using an enzyme immunosorbent assay (Invitrogen, Vienna, Austria): TNFα, IL-1β, IL-6, IL-17A and interferon (IFN)-gamma. The cell pellet was treated with triton X and IL-1α was measured in supernatants by an enzyme immunosorbent assay. CRP, troponin, NT-proBNP were measured in plasma with an immuno-chemiluminometric assay (ADVIA 1800/ADVIA 2400, Siemens Healthineers Laboratory Diagnostics, USA); and ferritin, IL-1Ra, IL-6 and calprotectin (S100A8/A9) with an enzyme immunosorbent assay (R&D Systems, Minneapolis, USA). Lower detection limits were: for CRP 0.5 mg/i; for troponin 12 ng/l; for NT-proBNP 35 pg/mL; for IL-1Ra and S100A8/A9 31 pg/mL; for TNFα 20 pg/mL; for IL-1β and IL-1α 10 pg/mL; for IL-6 40 pg/mL; for IFNγ 31 pg/mL; and for IL-17A 62 pg/mL.

### ChIP-Seq Sample Preparation and Analyses

Samples (n=7 for patients, n=5 for controls) were prepared for ChIP-seq using a previously published protocol (Agelopoulos, M., Thanos, D., EMBO J 25, 4843-4853, 2006) with modifications. Briefly, approximately 4 million PBMCs per sample were fixed with 1% formaldehyde for 30 minutes at room temperature and quenched with 0.125M glycine for 5 minutes. Following cell and nuclei lysis, chromatin was sonicated using a Covaris S220 instrument. For the immunoprecipitation step chromatin was incubated overnight with 6 μl of H3K27ac antibody (Cell signaling, #4353). Following immobilization on protein G beads and washing steps, samples were treated with proteinase K and crosslinks were reversed overnight at 65 C. The DNA was purified with Nucleomag beads and tested for enrichment with qPCR using specific primers. CHIP-sew libraries were prepared using the NEBNEXT® Ultra™ II DNA Library Prep Kit for Illumina. DNA concentration was measured with a Qubit fluorometer, and size was assessed with the Agilent bioanalyzer DNA1000 kit. Samples were sequenced in a NovaSeq 6000 Illumina sequencer at the Greek Genome Center in BRFAA.

Bioinformatics analyses were performed using The Galaxy suite (Afgan, E. et al., Nucleic Acids Res 46, W537-W544, 2018). The quality of the sequencing reads was evaluated using the FastQC algorithm. Sequencing reads were mapped to the hg19 version of the human genome using the Bowtie2 algorithm with the very sensitive end to end option (Langmead B., Salzberg S. L., Nat Methods 9, 357-359 2012). Duplicates were removed using the RmDup command from Samtools (Li, H. et al., Bioinformatics 25, 2078-2079, 2009). Peaks were called using the MACS2 algorithm with a q-value cut-off of 0.05 (Zhang, Y., et al., Genome Biol 9, R137, 2008). All peaks from the different samples were merged into a combined list in preparation for differential analysis. The MultiCovBed command from Bedtools (Quinlan, A. R., Hall, I. M., Bioinformatics 26, 841-842, 2010) was used to calculate reads for each sample that are contained in the combined list of peaks. Differential peaks between groups were calculated with the DESeq2 algorithm (Love, M. I. et al., Genome BioI 15, 550, 2014) using as a cut-off a log 2 foldchange of 0.4, a p value of 0.05 and a minimum count of 20 reads. Bigwig files were constructed using the bamCoverage command from the Deeptools suite (Ramirez, F., et al., Nucleic Acids Res 42, W187-191, 2014) and the scaling factors from DESeq2. Heatmaps were prepared with the computeMatrix and plotHeatmap commands from Deeptools. Gene ontologies for the closest genes to differential peaks were found using the GREAT tool (McLean, C. Y., et al., Biotechnol 28, 495-501, 2010) with the single closest gene (1000 kb) option. Gene ontologies for the closest genes to differential peaks were also analyzed using the Enrichr software (Chen, E. Y., et al., BMC Bioinformatics 14: 128, 2013). The genomic distribution of peaks was calculated using the CEAS software (Shin, H., et al., Bioinformatics 25, 2605-2606, 2009) from cistrome (Liu, T., et al., Genome Biol 12, R83, 2011). In order to perform motif analysis for regions with differential signal, we identified the accessible sub-regions under H3K27ac peaks which are known to host transcription factor binding. To do that we compiled a list of DNasel-seq accessible sites publicly available from the Encode (Encode project consortium, 2012) concerning the 4 major cell types of PBMCs and intersected that list with the differential H3K27ac sites. Motif analysis was carried out using the Meme-ChIP software (Machanick, P., Bailey, T. L., Bioinformatics 27, 1696-1697, 2011). To assess the similarity of our datasets with a compendium of publicly available DNase-seq and ChIP-seq experiments we used i-cisTarget.

### Endpoints

The study primary endpoint was the activation of innate immune response as assessed by the release of IL-1β and IL-6 from PBMCs after stimulation with LPS. The primary endpoint was evaluated in both cohorts. Secondary endpoints were evaluated only in the validation cohort. The secondary endpoints were: a) the production of Th1, Th2 and T17 cytokine responses; b) the incidence of PACS; and c) the association of cytokine production capacity with specific symptoms of PACS and/or LFTs.

### Statistical Analysis

Categorical data were presented as frequencies and confidence intervals (CI); continuous variables with normal distribution as mean with standard deviation (SD) and data with non-normal distribution as median with interquartile range. Fisher's exact test was used for comparison of categorical data whereas Student's t-test or non-parametric Mann Whitney test were used for the comparison of continuous data, as appropriate. Comparisons of the allocation of the groups of phenotypes of PACS between participants of the SAVE-MORE treated with placebo and with anakinra were done by ordinal regression analysis. Any two-sided p value lower than 0.05 was considered statistically significant. Statistical analysis was performed using the software IBM SPSS version 26.0.

### Data Availability

The sequence libraries generated in this study are publicly available through the National Center for Biotechnology Information (NCBI) gene expression omnibus (GEO) under the accession number GSE206605).

### Results

### Patients

Adult patients were prospectively enrolled in two cohorts 3-6 months after hospital discharge for COVID-19 pneumonia and negative molecular tests for SARS-CoV-2 at the time of enrolment (see FIG. 1). The discovery cohort was recruited from September to October 2020 and comprised 46 patients; they were compared with 25 matched comparators without known history of COVID-19 pneumonia and negative anti-SARS-CoV-2 antibodies. The validation cohort was recruited from patients who participated in the SAVE-MORE randomized clinical trial (Kyriazopoulou, et al., supra). In the SAVE-MORE trial, 594 patients with moderate-to-severe COVID-19 pneumonia were allocated to blind treatment with placebo and standard-of-care (SoC) treatment or anakinra and SoC. SoC contained dexamethasone administration for 10 days. 457 patients participated in the validation cohort at a median of 120 days post-hospital discharge; 135 patients originally received placebo; and 322 patients originally received anakinra. The follow-up of patients originally allocated to treatment with placebo is mirroring the time course towards PACS of patients with COVID-19. The validation cohort was analyzed as follows: a) patients were grouped into phenotypes of PACS based on the 16-symptoms questionnaire and the Symptom Tool-Impact Tool (ST-IT) questionnaire and the Short Form (SF)-36 healthy survey provided. PACS phenotypes derived from answers only from the 135 patients initially treated with placebo; b) we compared the distribution of phenotype groups of PACS between patients treated with placebo and patients treated with anakinra; c) we analyzed the response of circulating monocytes, as well as of Th1-, Th2- and Th17-responses of 96 placebo/SoC-treated COVID-19 patients and compared them with 21 matched healthy volunteer comparators; and d) we studied respiratory function through spirometry and carbon monoxide diffusing capacity (DL_{co}) in 60 patients earlier treated with placebo/SoC (FIG. 1). The demographics and comorbidities of patients and comparators were similar (Table 1).

TABLE 1
Demographics of the discovery and of the validation cohorts

| | Discovery cohort | | | | | Validation cohort | | | | |
|---|---|---|---|---|---|---|---|---|---|---|
| | Patients | | Comparators | | | Patients | | Comparators | | |
| | (n = 46) | | (n = 25) | | p | (n = 457) | | (n = 21) | | p |
| Days from | 120 | (89-180) | NA | | — | 140 | (120-210) | NA | | — |
| diagnosis, | | | | | | | | | | |
| median (Q1-Q3) | | | | | | | | | | |
| Moderate | 34 | (73.9) | NA | | — | 121 | (25.0) | NA | | — |
| disease, | | | | | | | | | | |
| n (%) | | | | | | | | | | |
| Severe disease, | 12 | (26.1) | NA | | — | 363 | (75.0) | NA | | — |
| n (%) | | | | | | | | | | |
| Dexamethasone | 12 | (26.1) | NA | | — | 363 | (75.0) | NA | | — |
| treatment on | | | | | | | | | | |
| acute COVID- | | | | | | | | | | |
| 19, n (%) | | | | | | | | | | |
| CCI, mean ± SD | 1.2 | (1.7) | 1.6 | (2.2) | 0.371 | 2.2 | (1.6) | 1.7 | (0.4) | 0.099 |
| Age in years, | 49 | (16) | 16 | (64) | 0.543 | 61 | (12) | 60 | (5) | 0.544 |
| mean (SD) | | | | | | | | | | |
| Male sex, n (%) | 30 | (62.2) | 16 | (64.0) | 1.00 | 261 | (54.0) | 9 | (43.0) | 0.085 |

NA: not applicable;
Q: quartile;
SD: standard deviation

### Primary Endpoint

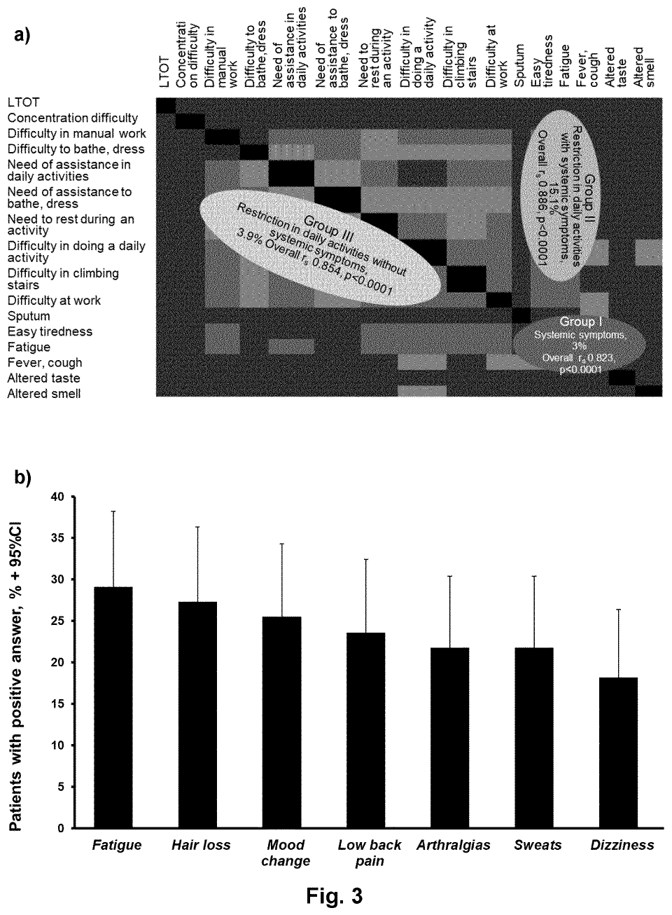

The study primary endpoint was the cytokine-production capacity of circulating immune cells following stimulation with bacterial lipopolysaccharide (LPS). The production capacity for both IL-1β and IL-6 of peripheral mononuclear blood cells (PBMCs) was significantly higher in patients recovering from COVID-19 pneumonia than comparators. This was independently validated in both the discovery cohort and the validation cohort (FIG. 2). Using receiver operator curve (ROC) analysis, it was found that a released concentration of IL-1β≥2500 pg/mL and of IL-6≥700 pg/mL could distinguish patients from comparators. The odds for IL-1β production greater than 2500 pg/mL among PACS patients was 5.1 (95% confidence intervals 1.4-18.4; p: 0.008). The respective odds for IL-6 production greater than 700 pg/mL was 8.5 (95% confidence intervals 2.9-24.5; p<0.0001).

### Post-Covid Symptoms in the Validation Cohort

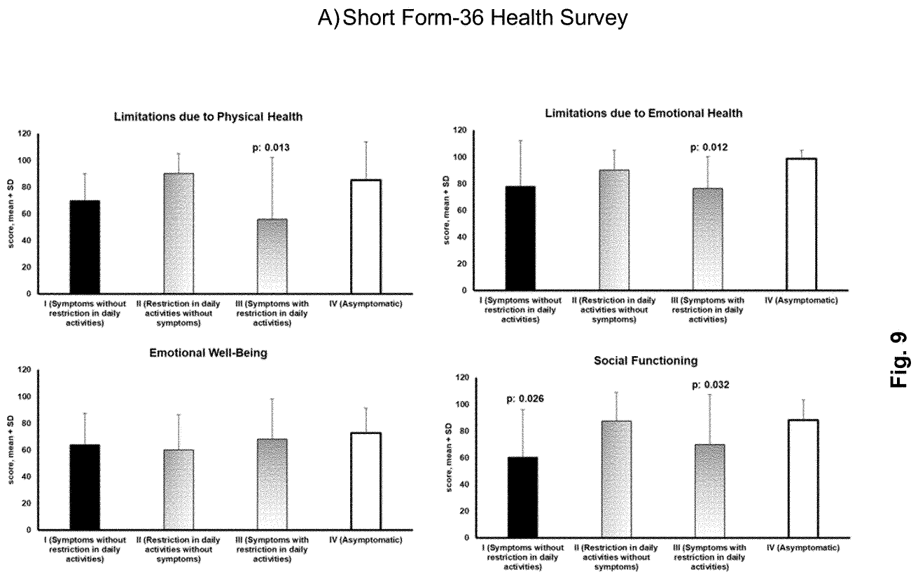

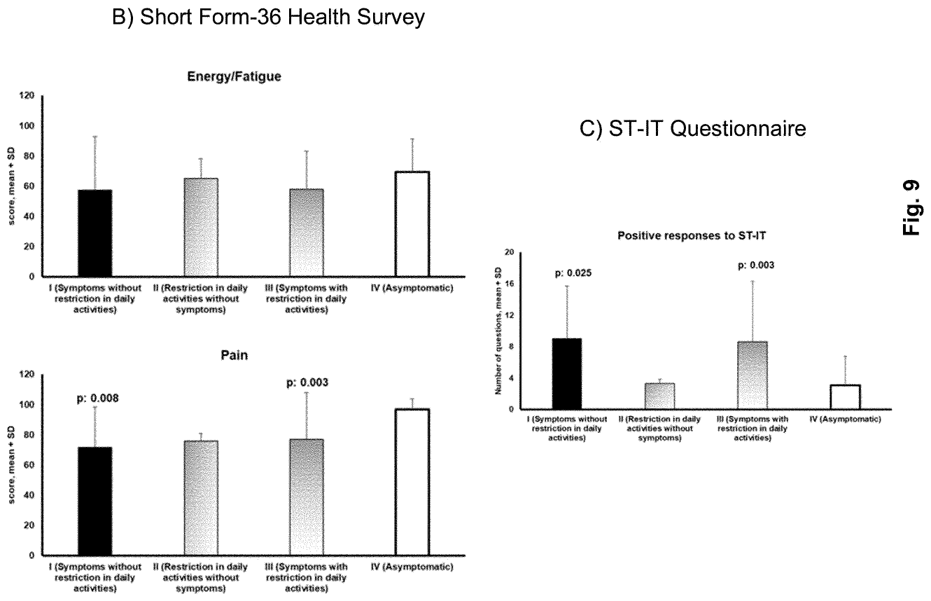

According to the study design, answers to 16-symptom-guided questionnaires were collected by patients of the validation cohort. This questionnaire was used as a tool of the evaluation of the quality of life in the SAVE-MORE trial. In order to define groups of PACS, we studied the 135 patients who were allocated to treatment with placebo; this allowed to avoid any interference of the administered treatment (anakinra). Analysis revealed four distinct groups of PACS: Group 1) systemic symptoms without restriction in daily activities (3%); Group II) restriction in daily activities with systemic symptoms (15.1%); Group 111) restriction in daily activities without systemic symptoms (3.9%); and Group IV) asymptomatic (78%). The total prevalence of PACS was 22% (FIG. 3a) with Group 111 being the most severe. The most common symptoms reported in the ST-IT questionnaire (6) were fatigue, hair loss, mood change, low back pain, arthralgias and sweats (FIG. 3b). Patients of Group III reported worse outcomes using in the SF36 health survey (FIGS. 9A-B) and greater number of symptoms in the ST-IT questionnaire (FIG. 9C).

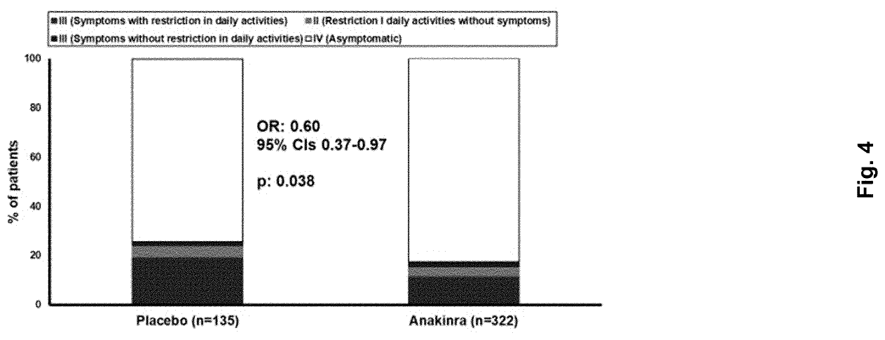

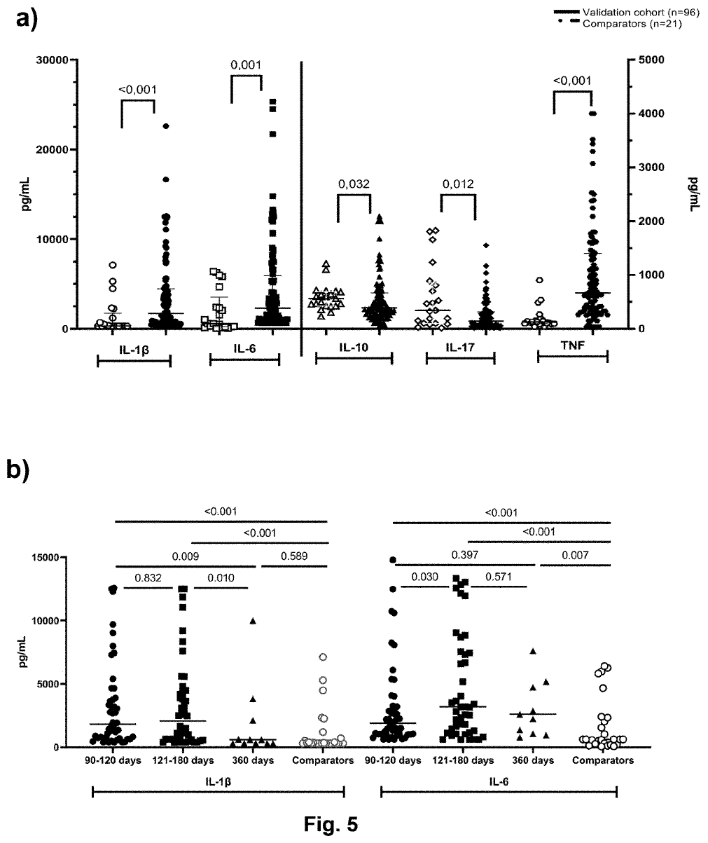

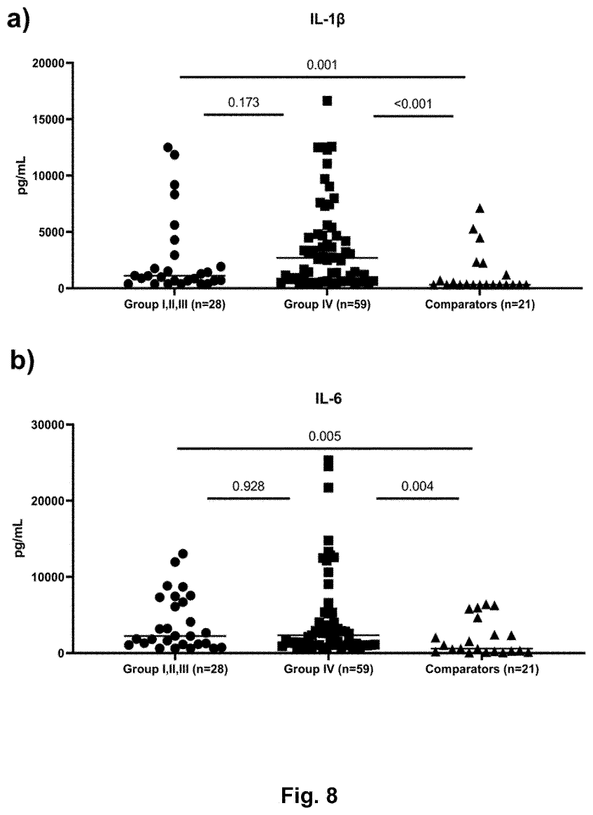

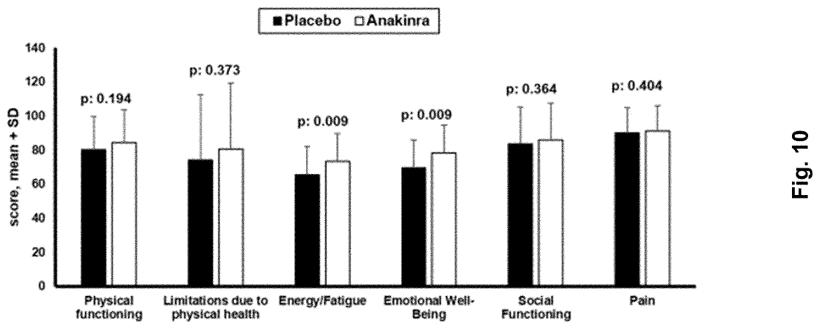

The classification of PACS into four groups generated two main questions: whether production of IL-1β and IL-6 was different in each group; and whether anakinra treatment modulated the development of these groups of PACS. The production of IL-1β, IL-6 and TNF following LPS stimulation was greater in all four groups than comparators. However, there were no differences between Groups I to IV (FIG. 8). This finding suggests that the modulation of circulating monocytes towards higher production of IL-1β and of IL-6 following recovery from pneumonia takes place even in the absence of symptoms. Anakinra treatment changed the allocation of grouping of PACS phenotypes. More precisely, patients who were treated with anakinra during acute illness had 0.60 odds for the development of the more severe Group III PACS phenotype compared to patients treated with placebo (FIG. 4). Patients treated with anakinra scored better in the elements of energy/fatigue and emotional well-being of the SF-36 health survey (FIG. 10).

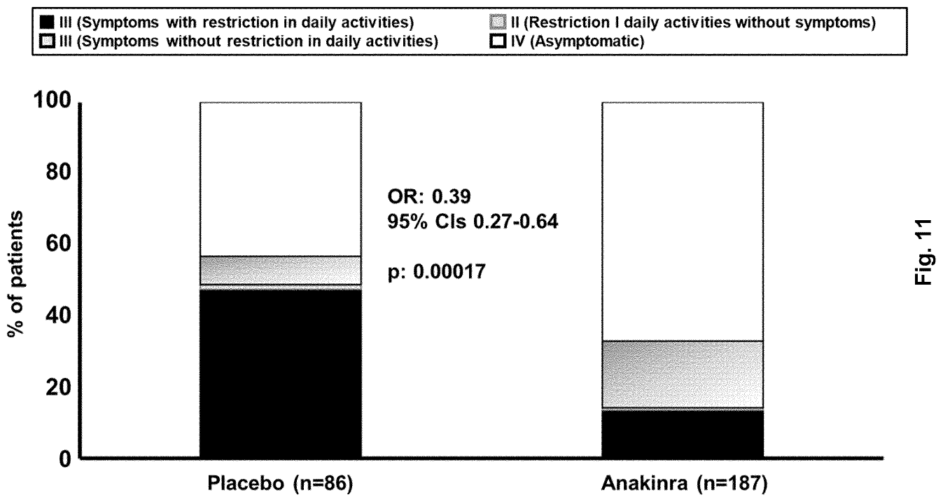

Patients of the validation cohort were contacted by phone calls one year after COVID-19 infection and were asked for their health status. To classify the status of the patients into each of the four groups of PACS, the same 16-element questionnaire was used. In parallel, patients were asked if they were diagnosed with any new disease or if they had to change treatment regimen for any disorder pre-existing COVID-19 or if they were in need of new hospitalization. 273 patients responded to the call of which 86 patients were originally allocated to treatment with placebo and 187 patients were originally allocated to treatment with anakinra. Forty-one patients (57.0%) and 64 patients (39.6%) were allocated to at least one of Groups I, II and III (p<0.0001) (FIG. 11). The incidence of new disorders was similar between the two groups (results not shown). However, nine (10.5%) and 7 (3.7%) of these patients were in need of new hospitalization (p: 0.048). This difference in the incidence of new hospitalizations was reflected among patients with the Group III phenotype of PACS; it was 18.2% among placebo-treated patients and 0% among patients treated with anakinra (results not shown).

### Inflammatory Mediators in Plasma and Th1/Th2/Th17 Responses

No differences were found in the circulating concentrations of NT-proBNP, troponin, C-reactive protein, ferritin, IL-18, interferon (IFN)-gamma and IL-1Ra between patients of the validation cohort and comparators. The only exception was the anti-inflammatory IL-33r which was decreased in post-COVID-19 patients (FIG. 24). The production of IL-10 and Th17 was also compared between patients of the validation cohort and comparators. TNFα (marker of Th1 responses) production was greater between post-COVID-19 patients than comparators; in contrast, IL-10 (marker of Th2 responses) and IL-17 (marker of Th17 responses) production was decreased in patients (FIG. 5a). In few patients, blood sampling was repeated 12 months after acute COVID-19; IL-1β and IL-6 production by circulating monocytes was decreased compared to the production measured before 180 days after acute illness, but it remained higher than comparators (FIG. 5b).

Association of Immune Dysregulation with Post-Covid Symptoms

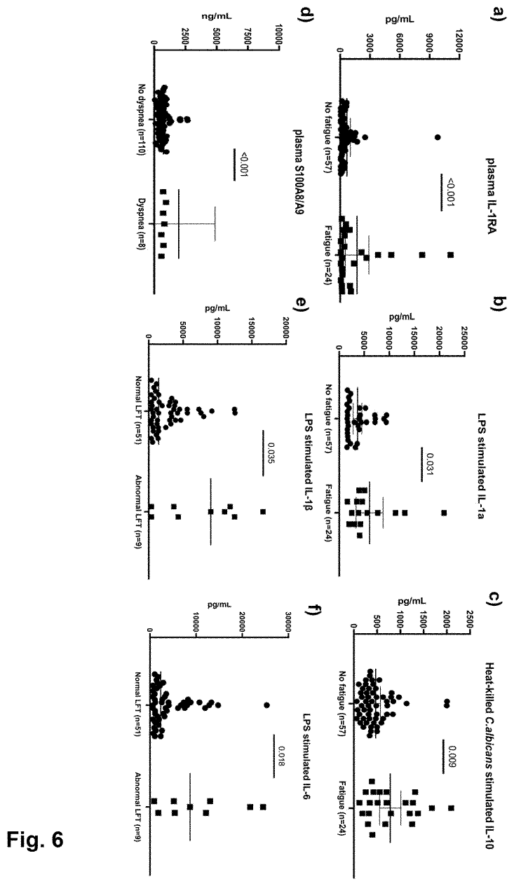

Next, we tried to associate the most common symptoms of PACS with cytokine production capacity. The most common symptoms of PACS in the validation cohort were fatigue and abnormal lung function tests. Patients after COVID-19 pneumonia experiencing fatigue had higher levels of circulating IL-1Ra, higher production of IL-1a after LPS stimulation of monocytes and higher IL-10 production from PBMCs when compared with patients not reporting fatigue (FIG. 6a, 6b, 6c). Exertional dyspnea which is indicative of lung disease was accompanied by increases of circulating calprotectin (S100A8/A9) (FIG. 5d). Among the 60 patients of the validation cohort who underwent lung function tests (LFT), nine patients presented with abnormalities compatible with interstitial lung disease. These were a) decrease of DL_{co} (defined as corrected values less than 76%) in four patients, and b) lung function restrictive pattern (ratio of forced expiratory volume in one minute to forced vital capacity [FEV1/FVC]<70%) in five patients. None of these patients had medical history of chronic respiratory disease. These patients had markedly increased IL-1β and IL-6 production capacity from monocytes after LPS stimulation (FIG. 6e, 6f).

### Long-Term Changes of the Epigenetic Histone Mark H3K27Ac

To investigate the molecular basis for the high-level expression of cytokines in PBMCs derived from LCS patients, we compared the genome-wide epigenetic modifications of histone H3K27 acetylation (H3K27Ac) in seven post-COVID-19 patients and five healthy controls. After ChIP-seq experiments to detect the distribution of H3K27ac in the human genome, we identified 708 regions with decreased H3K27ac levels in patient samples compared to control samples (results not shown). Genomic distribution analysis revealed an enrichment for promoters (10% of regions), while the vast majority of these were found either in distal intergenic or intragenic regions. To identify transcription factors that could participate in regulation of transcription from the above regions, we performed motif analysis and found that the motif for the master regulator of myeloid cells PU-1 is the most significant motif, followed by the Jun DNA binding motif, in agreement with the involvement of both transcription factors in immune processes. In addition, Gene ontology analysis using the GREAT algorithm indicated that the H3K27Ac regions are associated with genes involved in immune processes such as response to cytokine and response to lipopolysaccharide. For example, multiple regions bearing reduced H3K27ac levels in patient samples near the IL-1α gene were identified (results not shown).

Furthermore, we identified 422 regions with increased H3K27ac in patient samples (results not shown). Motif analysis identified the PU-1 binding site as the most significant DNA binding motif. However, no significantly enriched biological processes were associated for the genes closest to these regions using the GREAT algorithm. For example, the TRAF2 locus provides a representative region with increased H3K27ac levels in patient (results not shown).

### Discussion

The present clinical study demonstrated that a significant dysregulation of the capacity of monocytes and lymphocytes to produce cytokines persists long time after hospital discharge for COVID-19 pneumonia. This immune dysregulation lasts for up 25 to 12 months and occurs irrespective of the presence or absence of clinical symptoms of PACS. Major characteristics of this immune dysregulation are the increased IL-1β, IL-6 and TNF production by monocytes, accompanied by attenuated Th2 responses and T17 responses, while Th1 release of IFNg was largely normal.

PACS occurs in 22% of the patients and is characterized by considerable heterogeneity of symptoms. Although symptoms vary, the investigators identified associations between the prevalence of anti-inflammatory Th2 cellular response and fatigue, as well as between the increased IL-1β and IL-6 production by circulating monocytes and abnormalities in LFTs. The findings have been validated in two independent cohorts of patients that were compared with control volunteers without history of COVID-19 infection, who were matched for age, sex and comorbidities. The investigators also assessed the impact of anti-IL-1 therapy during the acute phase of the infection on the long-term symptoms post-COVID-19 in the SAVE-MORE clinical trial (Kyriazopoulou, et al., supra): patients originally allocated to SoC and anakinra treatment displayed significantly lower incidence of PACS compared with patients treated with CoC and placebo. The anakinra protection from PACS lasted as long as one year after recovery. These effects of anakinra on PACS suggest that over-production of IL-1β during the acute phase of the disease is likely to have an important impact on the development of PACS (Schultheiß, C. et al., Cell Rep Med 3, 100663, 2022).

The immune changes described here in post-COVID-19 patients share mixed features of auto-inflammatory syndromes, auto-immune syndromes and the post-intensive-care-unit syndrome (PICS), yet they remain distinct. More precisely, auto-inflammatory syndromes are characterized by increased monocyte activation with overproduction of IL-1β, but less so of IL-6 and TNF (Jamilloux, Y. et al., Rheumatology 57, 100-111, 2018). Also, it has been reported that the increased IL-1 production is associated with development of pulmonary fibrosis (Maekawa, T. et al., J Dermatol 40, 98-101, 2013; Colarusso, C. et al., Biomedicines 9: 1931, 2021). The imbalance towards a pro-inflammatory immune response due to T-cell function dysregulation is a common characteristic of auto-immune syndromes (Talaat, R. M. et al., Cytokine 72, 146-53, 2015; Ramos-Casals, M. et al., Semin Arthritis Rheum 32: 56-63, 2002). A link between immune dysregulation after COVID-19 and auto-immunity is suggested by the manifestation of fatigue, hair loss and arthralgias, which appear commonly in PACS but also in connective tissue disorders (Aringer, M. et al., Arthritis Rheumatol 71, 1400-1412, 2019). In a recent study of 31 patients with PACS, autoantibodies were also detected (Su Y, et al., Cell 185, 881-895.e20, 2022), arguing for common feature between PACS and autoimmune diseases. However, auto-immune diseases are generally accompanied by increased Th17 response, while in PACS this component of immune dysregulation seems to be absent (with even lower Th17 responses). Although PICS shares common clinical symptoms with PACS, over-activation of the immune response is not presented and clinical picture is framed from muscle asthenia and strenuous weaning from mechanical ventilation (Ramnarain, D. et al., Expert Rev Neurother 21, 1159-1177, 2021).

The current study also identified distinct relationships between specific components of immune dysregulation and the clinical characteristics of patients with PACS. In a scale of grading severity of immune dysregulation after recovery from COVID-19, the lowest level is represented by patients without any symptom and the highest level by those with persistent abnormal lung function. Fatigue may be considered as an intermediate level of severity. In the PACS patients described here, fatigue was associated by exaggerated Th2 immune responses and an increase in serum concentrations of IL-1Ra. However, it is important to underline that IL-1Ra is often associated with a high level of IL-1 bioactivity and represents a marker of inflammation (rather than the opposite). However, this inflammatory component seems to be relatively mild, as no increase in IL-1 production capacity has been identified in the monocytes of these patients. Alternatively, a source of IL-1β outside the circulating immune cells could be envisaged in these patients (e.g., tissue macrophages). Reversely, abnormalities in lung function were associated with aggravation of inflammation induced by monocytes and reflected by high circulating calprotectin levels. Calprotectin is a danger-associated molecular pattern (DAMP) released by lung epithelial cells during severe COVID-19 (Mahler, M. et al., Expert Rev Clin Immunol 17, 431-443, 2021), and circulating concentrations of calprotectin increase when COVID-19 progresses into critical illness (Kassianidis, G. et al., Int J Mol Sci 23, 4894, 2022). It has been suggested that calprotectin is recognized by Toll-like receptor (TLR)-4 on monocytes and can induce production of IL-1β and IL-6. A calprotectin-induced release of proinflammatory cytokines may explain why the incidence of PACS was lower among patients receiving anakinra during acute illness, since anakinra is blocking the excess production of IL-1 stimulated by calprotectin (Renieris, G. et al., J Innate Immun 1-14, 2022).

The findings that post-COVID-19 is characterized by increased cytokine production capacity leads to the question regarding the molecular mechanisms mediating these effects. Earlier studies have shown that infections (and certain vaccines) can induce long-term changes in cytokine production capacity of immune cells through epigenetically-mediated processes (DiNardo, A. R. et al., N Engl J Med 384, 261-270, 2021), a phenomenon named trained immunity. We hypothesized that a similar mechanism may result in long-term changes of immune function after COVID-19. Using ChIP-seq for H3K27ac as a marker of open chromatin at the level of both promoters and enhancers, we uncovered 708 regions in COVID-19 monocytes associated with decreased acetylation and 422 regions associated with increased acetylation. Many of the genes impacted by these changes have been shown to modulate the immune function of the host. The co-presence of regions of decreased and of increased acetylation may explain the complex immune dysregulation in post-COVID19 patients.

Our data are also supported by a number of recent studies from the literature. In a recent trial of 52 patients with hematologic malignancies, there was an increase after 3 months post-COVID-19 recovery of serum concentrations of cytokines, chemokines and growth factors that contribute to tumor growth and may be associated with unfavorable outcome of the underlying hematological disease (De Winter, F. H. R. et al., Cancers 13, 5718, 2021). Inflammatory changes in immune cells post-COVID19 have also been described at the level of production of interferons, and these changes persisted for up to 8-months (Phetsouphanh, C. et al., Nat Immunol 23, 210-216, 2021). These and our data argue for long-term dysregulation of immune responses after COVID-19.

In conclusion, patients recovering from COVID-19 pneumonia in need of hospitalization during acute infection present long-term post-infection immune dysregulation characterized by hyper-production of IL-1β and IL-6 from monocytes, and defects in Th2 and T17 responses. Exaggerated Th2 responses are associated with fatigue, while hyper-production of IL-1β and IL-6 is associated with abnormal lung function. Epigenetic modifications are associated with this dysregulation, and the persistently enhanced cytokine production could be designated as a state of trained immunity. Importantly, anti-IL-1 therapy with anakinra during the acute phase of the disease significantly decreased the incidence of PACS, arguing for the importance of innate immune dysregulation for the long-term effects of COVID-19. These data also argue for the necessity of prospective clinical trials of anti-inflammatory immunotherapy in patients with PACS.

### Example 2

### Clinical Study of PACS

### Aim of the Study

The following clinical study synopsis describes a phase II randomized clinical trial (SAVE-LONG) that aims to evaluate the efficacy and safety of the hIL-1R antagonist anakinra in patients with Post-Acute COVID Syndrome (PACS) in improving the clinical and immunological state over 4 to 8 weeks as measured by a composite endpoint, namely, the “Score of PACS progression reversal”.

### Methods

### Study Design

The study is a prospective double-blind, randomized clinical trial that takes place in European study sites. The study protocol is to be approved by the Institutional Review Boards (when required) and by the National Regulatory authorities. One general scheme of the study is provided in FIG. 7.

### Study Population

Patients who meet all the following inclusion criteria and who do not meet any of the following exclusion criteria are allowed to be enrolled:

### Inclusion Criteria

1. Age equal to or above 18 years.
2. Male or female gender.
3. In the case of women of childbearing age and men, an adequate method of contraception should be used during the study.
4. Written informed consent provided by the patient.
5. History of confirmed COVID-19 infection the last 90 days or more.
6. Symptoms compatible with PACS (defined as at least 3 out of 9 answers positive of a questionnaire for restriction of daily activities).
7. Presence of one of the following two clinical situations:

### Situation 1 Presence of:

Impaired Lung Function tests (defined as: DLCOcor <76% AND TLC and/or FVC lower than 80% of predicted).

### Situation 2 Presence of:

At least a total radiology score in HRCT more than 20 OR walking of a distance less than 500 m in the 6-minute walk test.

and

Abnormal immune function (defined as excessive IL-1β levels more than 2500 pg/ml after stimulation of peripheral blood mononuclear cells (PBMCs) with lipopolysaccharide (LPS).

If patients meet the criteria for both Situations 1 and 2, they will be considered for randomization and evaluation for the primary endpoint as in Situation 1.

Exclusion criteria
8. Age below 18 years
9. Denial for written informed consent
10. Any stage IV malignancy
11. Any primary immunodeficiency
12. Less than 1,500 neutrophils/mm3
13. Known hypersensitivity to anakinra
14. Known lung fibrosis prior to COVID-19
15. Known chronic obstructive pulmonary disease GOLD stage 3 or 4 prior to COVID-19
16. Known active tuberculosis (under treatment) or latent tuberculosis (by positive tuberculin test)
17. Oral or IV intake of corticosteroids at a daily dose equal or greater than 0.4 mg/kg prednisone for a period greater than the last 15 days.
18. Any anti-cytokine biological treatment the last one month; this includes treatment with anti-TNFs, soluble TNF receptors, anakinra, IL-6 receptor antagonists, anti-IL-17 and Janus kinase inhibitors
19. Severe hepatic failure defined as Child-Pugh stage of 3
20. End-stage renal failure necessitating hemofiltration or peritoneal hemodialysis
21. Pregnancy or lactation. Women of child-bearing potential will be screened by a urine pregnancy test before inclusion in the study
22. Participation in any other intervention

### Screening Procedure

The screening period may last up to 15 days. Screening is performed under the following steps:

Step 1: Patients are screened for the exclusion criteria. If they meet any of them, they cannot be enrolled. If they do not meet any of them, they remain eligible and screening proceeds to step 2. The following information is recorded at that stage: age and gender, height, weight, time since COVID-19 infection (days since positive RT-PCR test for SARS-CoV-2), need for hospitalization for COVID-19 in ward or intensive care unit and treatment received (i.e. dexamethasone, remdesivir, tocilizumab, baricitinib, anakinra or other), pre-existing comorbidities and chronic medical treatment.

Step 2: Patients are screened for symptoms compatible with PACS with the questionnaire for restriction of daily activities. If they answer positive in at least 3 of the 9 questions, they remain eligible and screening proceeds to step 3.

Step 3: A tuberculin test is performed. If tuberculin test is negative patients remain eligible and screening proceeds to step 4. For women of childbearing age, a pregnancy test is performed. These women remain eligible for the study only if the pregnancy test is negative.

Step 4: 20 ml of whole blood is drawn after venipuncture of one forearm vein under aseptic conditions for the conduct of a) complete whole blood cell counting (CBC); b) liver biochemistry, international normalized ratio (INR) and serum urea and creatinine; c) PBMC isolation and measurement of stimulated cytokine production. Lung function tests, i.e. spirometry and DLCO, lung HRCT and 6-minute walk test are performed. If patients meet the criteria of abnormal lung function tests as described in Situation 1 (DLCOcor <76% AND TLC and/or FVC lower than 80% of predicted) or have the combination of >20 total radiology score in lung HRCT or less than 500 m in 6-minute walk test with abnormal cytokine production after stimulation of PBMCs as described in Situation 2 (IL-1β levels more than 2500 pg/ml after stimulation of PBMCs with LPS) they may be enrolled in the study.

The total duration of the screening period cannot exceed 15 days. The lung function tests, the lung HRCT total radiology score, the 6-minute walk test result and the cytokine data from the stimulation of PBMCs obtained at that period are used to compare with the data from week 4 of enrolled patients.

### Intervention

Patients who meet all inclusion criteria and none of the exclusion criteria are allowed to be randomized in the study. Treatment with the study drug will comprise two periods, namely period A and period B. In period A, patients will be randomly assigned 1:1 to treatment Arm 1 and treatment Arm 2. A separate randomization computer-generated chart will be applied in each study site. Randomization will be stratified taking into consideration four strata:
Situation 1 and Situation 2
If the patient was hospitalized or not for COVID-19 infection; and in case of hospitalization if Intensive Care Unit (ICU) admission was needed
Treatment with dexamethasone or not during COVID-19 infection
Body mass index (>30 or ≤30 kg/m²)

The two groups of treatment in period A will be as follows:

Treatment Arm 1: patients receiving placebo. Placebo is injected subcutaneously once daily for 4 weeks.

Treatment Arm 2: patients receiving anakinra. Anakinra is injected subcutaneously as 100 mg once daily for 4 weeks.

The drug should be administered on the same time ±2 hours every day.

The extension period B will also have double blind characteristics. Patients allocated during period A to the placebo Treatment Arm 1 will be shifted to a subcutaneous treatment with 100 mg anakinra once daily for 4 weeks. Patients of Treatment Arm 2 of the first period will be randomized 1:1 to continue receiving subcutaneous treatment with 100 mg anakinra once daily for 4 weeks or placebo subcutaneously once daily for 4 weeks.

Concomitant medications are allowed during the study and they will be captured in the eCRF. The only NOT allowed concomitant medications are anti-cytokine biological treatments and more precisely:
Monoclonal antibodies targeting TNF (tumour necrosis factor-alpha) like infliximab adalimumab and certolizumab pegol
Soluble TNF receptors like etanercept
Monoclonal antibodies targeting TNF receptors like golimumab
Rilonacept and canakinumab
IL-6 receptor antagonists like tocilizumab, sarilumab and siltuximab
Monoclonal antibodies targeting IL-17 like secukinumab and bimekizumab
Monoclonal antibodies targeting IL-12 and IL-23 like ustekinumab and risenkizumab
Janus kinase inhibitors like baricitinib, abrocitinib, upatacitinib, and tofacitinib.

### Study Drug

Anakinra is the recombinant soluble antagonist of interleukin (IL)-1 receptor. This drug has strong anti-inflammatory potential by inhibiting both IL-1α and IL-1β. The mode of action of anakinra makes it a relatively safe drug since it acts through scavengering excess IL-1α and IL-1β and not by inhibiting basal production so as to introduce risk of infection for the patients.

Participants are allowed to self-inject with the investigational product or they may come to the study site to be injected. In case they decide to be self-injected, they will be trained by the study site staff how to self-inject but also on the selection of sites of injection. In each visit, patients will be given prefilled syringes in a cool box for transportation allowing the investigational product to remain at a temperature of 2-8° C. for at least two hours until this is safely placed in storage conditions of 2-8° C.

Pre-filled syringes containing placebo/anakinra will be provided by Sobi AB as kits containing 28 syringes.

### Patients' Visits

### Visits of Period A

Visit 1: This takes place at week 0 (day 1). The following are taking place during this visit:
Recording on demographics, time-period of confirmed COVID-19, past hospitalization for COVID-19, medical history, co-administered medication
Questionnaire for the presence of PACS
Quality of life questionnaire SF-36
Long Covid symptom tool and Impact tool questionnaire ST-IT
Visual analogue scale (VAS) scoring by the patient overall for PACS
VAS scoring by the patient overall for fatigue
Systolic and diastolic blood pressure, heart rate, oxygen saturation in room air, breath rate and core temperature
Electrocardiogram and echocardiogram with estimation of the left ventricle ejection fraction (LVEF) and pulmonary artery pressure
Providing the patient 28 prefilled syringes for self-injection.

Visit 2: This visit is a phone visit and takes place at week 2 (day 14±2). The following are taking place during this visit:
Recording of co-administered medication
Questions for any treatment-emergent adverse event (TEAE)
Questionnaire for the presence of PACS
Quality of life questionnaire SF-36
Long Covid symptom tool and Impact tool questionnaire ST-IT
Visual analogue scale (VAS) scoring by the patient overall for PACS
VAS scoring by the patient overall for fatigue

Visit 3: This takes place at week 4 (day 28±2). The following are taking place during this visit:
Accountability of the empty syringes being self-administered or any unused syringes
Recording of co-administered medication
Questions for any treatment-emergent adverse event (TEAE)
Questionnaire for the presence of PACS
Quality of life questionnaire SF-36
Long Covid symptom tool and Impact tool questionnaire ST-IT
Visual analogue scale (VAS) scoring by the patient overall for PACS
VAS scoring by the patient overall for fatigue
Systolic and diastolic blood pressure, heart rate, oxygen saturation in room air, breath rate and core temperature
Intake of 10 ml of blood after venipuncture of one antecubital vein under sterile conditions for the conduct of safety lab tests
Intake of 10 ml of blood after venipuncture of one antecubital vein under sterile conditions for PBMC isolation and measurement of stimulated cytokine production
Electrocardiogram and echocardiogram with estimation of the left ventricle ejection fraction (LVEF) and pulmonary artery pressure
High-resolution chest computed tomography (HRCT) with individual scoring of each of the type of lesions
Six-minute walk test
Delivering the patient 28 prefilled syringes for self-injection for the extension period.

Visit 4: This visit is a phone visit and takes place at week 6 (day 42±2). The following are taking place during this visit:
Recording of co-administered medication
Questions for any treatment-emergent adverse event (TEAE)
Questionnaire for the presence of PACS
Quality of life questionnaire SF-36
Long Covid symptom tool and Impact tool questionnaire ST-IT
Visual analogue scale (VAS) scoring by the patient overall for PACS
VAS scoring by the patient overall for fatigue

Visit 5: This takes place at week 8 (day 56±2). The following are taking place during this visit:
Accountability of the empty syringes being self-administered or any unused syringes
Recording of co-administered medication
Questions for any treatment-emergent adverse event (TEAE)
Questionnaire for the presence of PACS
Quality of life questionnaire SF-36
Long Covid symptom tool and Impact tool questionnaire ST-IT
Visual analogue scale (VAS) scoring by the patient overall for PACS
VAS scoring by the patient overall for fatigue
Systolic and diastolic blood pressure, heart rate, oxygen saturation in room air, breath rate and core temperature
Electrocardiogram and echocardiogram with estimation of the left ventricle ejection fraction (LVEF) and pulmonary artery pressure
High-resolution chest computed tomography (HRCT) with individual scoring of each of the type of lesions
Six-minute walk test

For all study visits, questionnaires will be filled out by patients through a phone application in their language. Questionnaires to be filled out in app are:
Questionnaire for the presence of PACS
Quality of life questionnaire SF-36
Long Covid symptom tool and Impact tool questionnaire ST-IT
Visual analogue scale (VAS) scoring by the patient overall for PACS
VAS scoring by the patient overall for fatigue

### Laboratory Procedures

Blood samples coming from screening visit and study visit 3 will be collected as follows: a) 3 ml into one EDTA coated tubes for complete blood cell counting; b) 2.5 ml into one sterile tube for complete biochemistry; c) 4 ml into one heparin-coated tube for coagulation tests and proteomic analysis; and d) 10 ml into one EDTA coated tubes for PBMC isolation and cytokine production measurement. Samples will be shipped from study sites into the study central lab that will be the Laboratory of Immunology of Infections at the 4^{th} Department of Internal Medicine at ATTIKON University General Hospital or any other subcontracting lab collaborating with the Sponsor.

The following safety complete cell counting tests will be done:
Total red blood cell count, MCV, MCH and MCHC
Total white blood cell count and differential
Total platelet cell count

The following safety biochemistry lab tests will be done:
Glucose, urea, creatinine
AST, ALT, yGT, SAP, total bilirubin and direct bilirubin
Electrolytes (Na, K, Ca)

The following safety coagulation lab tests will be done:
INR, aPTT, fibrinogen
D-dimers

PBMCs will be isolated and cytokine stimulation capacity will be assessed for the synthesis of monocyte-driven cytokines, Th1-driven cytokines, Th2-driven cytokines, and T17-driven cytokines. Plasma will be used for full proteomics analysis.

### Analysis of Radiological Findings

Each of the HRCT films will be scored by two independent experts who are completely blind to the allocation of treatment arm for each of the following type of lesions:
ground-glass opacities (GGO), consolidation, reticulation and honeycomb-like changes. Each of these four types of lesions will receive a separate score ranging from 0 to 5 for every of the five lung lobes denoting the level of involvement of each lobe as follows (Han X et al., Radiology 2021; 299: E177-E186):
0: no involvement
1: less than 5% involvement
2: 5-25% involvement
3: 26-49% involvement
4: 50-75% involvement
5: greater than 75% involvement

As such, a separate score is generated ranging from 0 to 25 for each of GGO, consolidation, reticulation and honeycomb-like changes. The definition for each of the radiological changes is as follows (Ito Y et al., Mod Rheumatol 2019; 29: 98-104):

GGO: increased parenchymal density with preservation of the bronchial and vascular markings, with or without very fine texture superimposed, but without obvious reticulation.

Consolidation: a homogeneous increase in pulmonary parenchymal density obscuring the underlying vessels.

Reticulation: crisscrossing linear opacities that were fine or coarse (including interlobular septal or intralobular septal thickening), with associated distortion of the lung architecture.

Honeycomb: air-filled cystic spaces with irregular walls deemed not to represent traction bronchiectasis.

The total score of fibrosis is the sum of scoring for reticulation and honeycomb-like lesions. The total radiological lung score is the sum of individual scores.

For the needs of the analysis, the files of the HRCT are sent for grading to the central lab appointed by the Sponsor.

### Primary Study Endpoint

A composite primary endpoint will be applied. This endpoint will be named “Score of PACS progression reversal”. A positive score is defined differently for patients enrolled in the study because of Situation 1 or Situation 2. For patients enrolled in the study because of Situation 1, a positive score comprises at least two of the following evaluation elements:
At least 20% improvement of restrictive lung disease (increase of at least one of DLCOcor, TLC, FVC or FEV1/FVC) from baseline
No need for hospitalization or admission to the Emergency Department for any cause
No increase of the degree of lung fibrosis score in lung HRCT

For patients enrolled in the study because of Situation 2, a positive score comprises at least two of the following evaluation elements:
At least 20% decrease of the total radiology score in lung HRCT OR Improved exercise capacity (defined as more than 30 meters improvement) in the 6 min walk test (depending on which of the two criteria the patient was enrolled)
No need for hospitalization or admission to the Emergency Department for any cause
No increase of the degree of lung fibrosis score in lung HRCT

The proportion of patients achieving the above composite endpoint compared to placebo at week 4 will be the primary study endpoint.

### Rationale for the Development of the Criteria of the Primary Endpoint

In order to develop the trial endpoints, a thorough search was done at PubMed database which contained the following search terms:
Spirometry AND long COVID AND clinical trials
Lung computed tomography AND long COVID AND clinical trials
Lung computed tomography AND long COVID
Echocardiography AND long COVID

The idea was to collect for each of the elements of the primary and of the key secondary endpoints, the chance for improvement without therapy over-follow-up. The reason for selecting the specific three elements to build the primary endpoint is that all these elements are reported by at least three other publications (Han X et al., Radiology 2021; 299: E177-E186; Bellan M et al., Sci Rep 2021; 11: 22666; Steinbeis F et al., Respir Med 2022; 191:106709; Shah A S et al., Thorax 2022; 76: 402-404; Fortini A et al., Infection. 2022; 50:513-517). The two key secondary endpoints are selected because they are reported by at least one publication (Han X et al., Radiology 2021; Romero-Duarte Á et al., BMC Med 2021; 19: 129; Nopp S et al., Respiration 2022; 101: 593-601). For the change of the LVEF and for the pressure of the pulmonary artery, no study could be found reporting on these outcomes; these two endpoints were provided as secondary endpoints. All selected cut-off of improvements in the proposed elements of both the primary and key secondary endpoints are greater than the published chance of spontaneous improvement.

### Secondary Study Endpoints

The comparison of the following between the two arms of treatment will be the study secondary endpoints:
The frequency of the Score of PACS progression reversal between patients receiving 8 weeks anakinra treatment compared to patients receiving 4 weeks anakinra treatment (+4 weeks of placebo)
Changes in cytokine production capacity of stimulated PBMCs at week 4 between the two arms of treatment
Change of each component of the score for the primary outcome at week 4 between the two arms of treatment
At least 10% decrease of the pulmonary artery pressure at week 4 between the two arms of treatment
At least 10% increase of LV ejection fraction (if abnormal at baseline) at week 4 between the two arms of treatment
Safety of anakinra

### Exploratory Study Endpoints

Changes of the number of answers from positive to negative in the questionnaire of the restriction of daily activities at week 4 between the two arms of treatment
Changes of the number of answers from positive to negative in the questionnaire of the restriction of daily activities between patients receiving 8 weeks anakinra treatment compared to patients receiving 4 weeks anakinra treatment (+4 weeks of placebo)
Changes of the number of answers from positive to negative in the ST/IT questionnaire at week 4 between the two arms of treatment
Changes of the number of answers from positive to negative in the ST/IT questionnaire between patients receiving 8 weeks anakinra treatment compared to patients receiving 4 weeks anakinra treatment (+4 weeks of placebo)
Changes of the responses to the questions of SF36v at week 4 between the two arms of treatment
Changes of the responses to the questions of SF36v at week 8 between patients receiving 8 weeks anakinra treatment compared to patients receiving 4 weeks anakinra treatment (+4 weeks of placebo)
Changes of the Global VAS for PACS at week 4 between the two arms of treatment
Changes of the Global VAS for PACS at week 8 between patients receiving 8 weeks anakinra treatment compared to patients receiving 4 weeks anakinra treatment (+4 weeks of placebo)
Changes of the VAS for fatigue at week 4 between the two arms of treatment
Changes of the VAS for fatigue at week 8 between patients receiving 8 weeks anakinra treatment compared to patients receiving 4 weeks anakinra treatment (+4 weeks of placebo)

### Number of Patients

The needed number of patients is based on the following hypothesis:
The placebo arm will show a 20% response in the composite primary endpoint, based on the average of results for the published chances of spontaneous improvement of PACS.
The anakinra arm will show a 36% response in the composite primary endpoint
80% power at the 5% level of significance are used
1:1 randomization applies
10% drop-outs

Final calculation: 182 patients need to be enrolled in total (91 patients in Arm 1; and 91 patients in Arm 2).

### Statistical Analysis

The analysis of the primary study endpoint will be done according to the ITT principle including the Intent-to-Treat (ITT) population by logistic regression and expressed as an odds ratio for improved outcomes (with respective 95% Confidence Interval).

Imputation of missing data will be done by the principle of Last Observation Carried Forward (LOCF). In this analysis the last measured value of the endpoint is imputed to all subsequent, scheduled, but missing, evaluations. The primary endpoint will be verified after forward logistic regression analysis using as co-variates the four strata of stratified randomization was done i.e.
Situation 1 and Situation 2
If the patient was hospitalized or not for COVID-19 infection; and in case of hospitalization if Intensive Care Unit (ICU) admission was needed
Treatment with dexamethasone or not during COVID-19 infection
Body mass index (>30 or ≤30 kg/m²)

Two sensitivity analyses of the primary endpoint will be done:
Per-protocol (PP) analysis including only patients who completed the entire 28 self-injections during the study period A.
One analysis imputing all missing data as failures.

Any p-value below 0.05 will be considered significant.

### Results

It is expected that administration of anakinra as described above will result in a significant improvement of PACS patient outcomes, in comparison with the control group. Significant improvements are expected in one or several of the endpoints listed above. The inventive treatment is expected to be tolerated well and fulfill safety requirements.

### Example 3

### Clinical Study of PACS

### Aim of the Study

The PRECISION is a proof-of-concept, phase II randomized clinical trial aiming to evaluate the efficacy and safety of anakinra in patients with Post-Acute COVID Syndrome (PACS) of the pro-inflammatory respiratory phenotype. Improvement is measured by a composite endpoint, namely, the “Score of PACS progression reversal”.

### Methods:

### Study Design

Prospective multicenter, double-blind, randomized clinical trial with an extension period.

### Study Phase

2/3

### Study Population

Patients who meet all the following inclusion criteria and who do not meet any of the following exclusion criteria are allowed to be enrolled:

### Inclusion Criteria

1. Age equal to or above 18 years
2. Male or female gender
3. In the case of women of childbearing age and men, an adequate method of contraception should be used during the study. Contraception should be maintained for at least a period of 3 months after the discontinuation of treatment. Prior to admission to the study, a pregnancy test will be performed to exclude pregnancy to women of childbearing age.
4. Written informed consent provided by the patient.
5. History of confirmed COVID-19 infection in the last 90 or more days
6. Symptoms compatible with PACS (defined as at least one positive answer to the questionnaire for restriction of daily activities) lasting for more than 2 months
7. Serum levels of IP-10 more than 250 pg/ml
8. Presence of ONE of the following two clinical conditions:

Condition 1: Impaired Lung Function tests (defined as: DLCOcor <76% AND TLC and/or FVC lower than 80% of predicted)

Condition 2: At least a total radiology score in HRCT more than 20 OR walking of a distance less than 500m in the 6-minute walk test.

If patients meet the criteria for both Conditions 1 and 2, they will be considered for randomization and evaluation for the primary endpoint as in Condition 1.

### Exclusion Criteria

Age below 18 years
Denial for written informed consent
Any stage IV malignancy
Any primary immunodeficiency
Less than 1,500 neutrophils/mm³
Known hypersensitivity to anakinra
Known lung fibrosis prior to COVID-19
Medical history of pulmonary hypertension or chronic heart failure
Known chronic obstructive pulmonary disease GOLD stage 3 or 4 prior to COVID-19
Known active tuberculosis (under treatment) or latent tuberculosis (by positive tuberculin test)
Oral or IV intake of corticosteroids at a daily dose equal or greater than 0.4 mg/kg prednisone for a period greater than the last 15 days.
Any anti-cytokine biological treatment the last one month
Severe hepatic failure defined as Child-Pugh stage of 3
End-stage renal failure necessitating hemofiltration or peritoneal hemodialysis
Pregnancy or lactation. Women of child-bearing potential will be screened by a urine pregnancy test before inclusion in the study
Participation in any other interventional trial

### Screening Procedure

The screening period may last up to 15 days. Screening is performed under the following steps:
Step 1-3: As described for Step 1-3 in Example 2.
Step 4: 20 ml of whole blood is drawn after venipuncture of one forearm vein under aseptic conditions for the conduct of a) complete whole blood cell counting (CBC); b) liver biochemistry, international normalized ratio (INR) and serum urea and creatinine; c) measurement of IP-10. Lung function tests, lung HRCT and 6-minute walk test are also performed. If patients have IP-10 more than 250 pg/ml and meet the criteria of at least one of conditions 1 and 2 described in inclusion criterion 1, they may be enrolled in the study.

The total duration of the screening period cannot exceed 15 days. The lung function tests, the lung HRCT total radiology score, the 6-minute walk test result and the IP-10 levels obtained at that period are used to compare with the data from week 4 of enrolled patients.

### Intervention

Patients who meet all inclusion criteria and none of the exclusion criteria are allowed to be randomized in the study. Treatment with the study drug will comprise two periods, the double-blind randomized period A and the extension period B. In period A, patients will be randomly assigned 1:1 to treatment Arm 1 and treatment Arm 2. A separate randomization computer-generated chart will be applied in each study site.

Randomization will be stratified taking into consideration four strata:
Condition 1 or Condition 2
Whether the patient was hospitalized for COVID-19 infection; and in case of hospitalization, if Intensive Care Unit (ICU) admission was needed (sub-stratification applies for patients hospitalized in the ICU).
Treatment with dexamethasone or not, during acute COVID-19 infection
Body mass index (>30 or ≤30 kg/m²)

The two groups of treatment in period A will be as follows:

Treatment Arm 1: patients receiving placebo. Placebo is injected subcutaneously once daily for 4 weeks.

Treatment Arm 2: patients receiving anakinra. Anakinra is injected subcutaneously as 100 mg once daily for 4 weeks.

The drug should be administered on the same time ±2 hours every day.

The extension period will also have double blind characteristics. Patients allocated during period A to the placebo Treatment Arm 1 will be shifted to subcutaneous treatment with 100 mg anakinra once daily for 4 weeks. Patients of Treatment Arm 2 of the first period will be randomized 1:1 to continue receiving subcutaneous treatment with 100 mg anakinra once daily for 4 weeks or placebo once daily for 4 weeks.

In accordance with Example 2, concomitant medications are allowed during the study and they will be captured in the eCRF. The only concomitant medications that are not allowed are the ones described in Example 2.

### Study Drug

Anakinra, as described in Example 2. The dose selected for this study is 100 mg subcutaneously once daily. This dose was selected based on the existing safety data since greater daily doses increase the risk for adverse events.

In accordance with Example 2, participants are allowed to self-inject with the investigational product or they may come to the study site to be injected. Each patient will be given a calendar to fill in the date and time of injection and the place of injection. At each visit the patient needs to provide the empty and any unused syringes to the site staff.

Pre-filled syringes containing placebo/anakinra will be provided by Sobi AB as kits containing 28 syringes.

### Patients' Visits

Visit 1: In accordance with Visit 1 of Example 2, this takes place at week 0 (day 1). In addition to what is described in Example 2, Visit 1 involves blood sampling of 14 ml for the isolation and culture of PBMCs.

Visit 2: This takes place in accordance with what is described for Visit 2 in Example 2.

Visit 3: This takes place in accordance with what is described for Visit 3 in Example 2. In addition, lung function tests (Spirometry, plethysmography, DLco) are carried out during Visit 3.

Visit 4: This visit is a phone visit and is carried out as described for Visit 4 in Example 2.

Visit 5: This takes place in accordance with what is described for Visit 5 in Example 2. In addition, lung function tests (Spirometry, plethysmography, DLco) are carried out during Visit 5.

In accordance with what is described in Example 2, questionnaires will be filled out by patients for all study visits.

### Laboratory Procedures

Laboratory procedures are carried out as described in Example 2 apart from the following: Blood samples coming from study visit 1 will be collected as follows: a) 4 ml into one heparin-coated tube for coagulation tests and proteomic analysis; and b) 10 ml into one EDTA coated tubes for PBMC isolation and cytokine production measurement.

Blood samples coming from study visit 3 will be collected as follows: a) 3 ml into one EDTA coated tube for complete blood cell counting; b) 3 ml into one sterile tube for complete biochemistry; c) 4 ml into one heparin-coated tube for coagulation tests and proteomic analysis; and d) 10 ml into one EDTA coated tube for PBMC isolation and cytokine production measurement.

### Analysis of Radiological Findings

Analysis of radiological findings is carried out as described in Example 2.

### Primary Study Endpoint

The primary study endpoint is the same as described in Example 2. However, in this study “Situation 1” corresponds to “Condition 1” and “Situation 2” corresponds to “Condition 2”.

### Secondary Study Endpoints

The secondary study endpoints are the same as those described in Example 2.

### Exploratory Study Endpoints

The Exploratory study endpoints are the same as those in Example 2.

### Number of Patients

The needed number of patients is based on the same hypothesis as in Example 2. The calculated number of patients is the same as that in Example 2.

### Statistical Analysis

Statistical analysis is carried out in accordance with the statistical analysis of Example 2.

### Results

It is expected that administration of anakinra as described above will result in a significant improvement of PACS patient outcomes, in comparison with the control group. Significant improvements are expected in one or several of the endpoints listed above. The inventive treatment is expected to be tolerated well and fulfill safety requirements.

### Example 4

### Lona-Term Immune and Epigenetic Dysregulation Following Covid-19

Since acute COVID-19 is dominated by immune dysregulation, e.g. hyperinflammation mediated by IL-1 and IL-6, it is reasonable to hypothesize that persisting immune dysregulation may also be an underlying mechanism of PACS. The immune dysregulation of IL-1 and IL-6 production in patients recovering from COVID-19 pneumonia requiring hospitalization in the acute phase was therefore investigated, as well as their involvement in the pathogenesis of PACS. The impact of inhibiting the excessive inflammation in the acute phase of the disease on the incidence of PACS was evaluated within the phase 3 SAVE-MORE clinical trial patients which showed the clinical efficacy of anakinra treatment for acute COVID-19 pneumonia (Kyriazopoulou, et al, supra).

### Methods

### Study Design

This study was prospective, observational and it was conducted in two outpatient post-covid units in Greece.

### Inclusion Criteria

a) adults aged 18 years or older; b) history of hospitalization for COVID-19 pneumonia the last 3 to 6 months; and c) negative molecular test for SARS-CoV-2 (Cobas SARS-COV-2 RT-PCR, Roche Diagnostics).

### Exclusion Criteria

a) any stage IV malignancy; b) any primary immunodeficiency; c) neutropenia with fewer than 1,500 neutrophils per mm³; d) oral or intravenous intake of corticosteroids at a daily dose greater than or equal to 0.4 mg kg⁻¹ of prednisone or equivalent for a period longer than 15 consecutive days the last 3 months; and e) any chronic anti-cytokine biological treatment.

### Methods

Adult patients were prospectively enrolled in three cohorts 3-6 months after hospital discharge for COVID-19 pneumonia and negative molecular tests for SARS-CoV-2 at the time of enrolment. The discovery cohort was recruited from September to October 2020 and comprised 46 patients; they were compared with 25 sex- and age-matched comparators without known history of COVID-19 infection and negative anti-SARS-CoV-2 antibodies. The validation cohort 1 was recruited from patients who participated in the SAVE-MORE randomized clinical trial. In the SAVE-MORE trial, 594 patients with moderate-to-severe COVID-19 pneumonia were allocated to blind treatment with placebo and standard-of-care (SoC) treatment or anakinra and SoC for their pneumonia. SoC contained dexamethasone and remdesivir administration for 10 and 5 days respectively. 464 patients participated in the validation cohort 1 at a median of 120 days post-hospital discharge; 137 patients were treated with placebo; and 327 patients treated with anakinra.

The validation cohort 2 was derived from 160 consecutive admissions in an out-patient infectious diseases unit 120 days post discharge for COVID-19 pneumonia.

From patients of both cohorts, 20 ml of blood sample was drawn from a peripheral forearm vein under aseptic conditions. The samples were collected into a) tubes with ethylenediaminetetraacetic acid (EDTA) and transferred within one hour to the central laboratory for isolation and culture of peripheral blood mononuclear cells (PBMCs) and b) in sterile pyrogen-free tubes for serum collection. For all patients, the following information was collected: date of COVID-19 infection, severity of disease, demographics (age, sex) and Charison's comorbidity index (CCI). Similar blood sampling was repeated in some patients of the validation cohort 12 months after hospitalization for COVID-19 pneumonia and in comparators without known history of COVID-19 infection, irrespective from vaccination status and matched for age, sex and CCI.

Patients of the validation cohorts were requested to provide answers to three different questionnaires; the first contained 16 questions to be answered as either Yes or No and developed by the researchers as a tool of quality of life used in the SAVE-MORE trial; the second was the ST-IT questionnaire which is internationally proposed for follow up of COVID-19 patients (Tran, V. T., et al, Clin Infect Dis 74, 278-287, 2022); and the third was the SF-36 health survey. The 16 questions of the first questionnaire concerned the presence one or more of the following: long-term home oxygen therapy, difficulty in concentration, need for assistance in everyday activities, need to rest during an activity, difficulty in doing a daily activity, dyspnea, fever/cough, sputum, difficulty in manual work, difficulty in climbing stairs, easy tiredness, feeling of fatigue, difficulty to bathe or dress, altered taste and smell. Patients with history of COVID-19 infection were classified as PACS if they answered positive at least one question of the 16-symptom questionnaire and if the symptom could not be explained by an alternative diagnosis. To exclude an alternative diagnosis, patients underwent thorough clinical, laboratory and radiology evaluation by their attending physicians.

In patients of the validation cohorts lung function tests were performed including spirometry, plethysmography and diffusing capacity for carbon monoxide (Elite Series Plethysmograph, Medical Graphics, Minessota, USA).

PBMCs were isolated using gradient centrifugation over Ficoll-Hypaque density gradient (Biochrom, Berlin, Germany) after 1400 g under 4° C. centrifugation over 25 minutes. After three washings in ice-cold PBS (phosphate buffered saline, pH: 7.2, Biowest, France), PBMCs were counted using a Neubauer plate with trypan blue exclusion of dead cells. PBMCs were incubated in 1 mL RPMI 1640 (Biochrom, Berlin, Germany) enriched with 100 U/ml penicillin G and 0.1 mg/ml streptomycin and distributed on 96-well plates at final volume 200 μL and density 5×10⁶/ml. Cells were incubated at 37° C. and 5% CO₂ for 24 hours without/with 10 ng/ml lipopolysaccharide (LPS) of Escherichia coli O55:B5. Cells were also incubated in same circumstances for five days with 10% fetal bovine serum without/with 5×10⁶ cfu/ml heat-killed Candida albicans (HKCA). After incubation the supernatants were collected. The cell pellet was treated with triton X and IL-1α was measured in supernatants by an enzyme immunosorbent assay. CRP, troponin, NT-proBNP were measured with an immuno-chemiluminometric assay (ADVIA 1800/ADVIA 2400, Siemens Healthineers Laboratory Diagnostics, USA) in plasma. Cytokines and inflammatory mediators were measured in plasma and cell supernatants by enzyme immunosorbent assays. Lower detection limits were: for CRP 0.5 mg/i; for troponin 1 2 ng/l; for NT-proBNP 35 pg/ml; for IL-1ra and S100A8/A9 31 pg/ml; for TNF 20 pg/ml; for FGF23 156pg/ml; for IL-1β and IL-1α 10 pg/ml; for IL-6 40 pg/ml; for IL-10 25 pg/ml; for IL-17 8 pg/ml; for IL-22 16 pg/ml; for IL-33r 1565 pg/ml; for IFNγ 78 pg/ml; for IP-10 63 pg/ml; for MMP-9 16 ng/ml; for PDGF-A 312 pg/ml; for sgp130 15.5 ng/ml; for s-Selectin 15 ng/ml; for S100A8/A9 63 ng/ml.

Samples (n=7 for patients, n=5 for controls) were prepared for ChIP-seq using a previously published protocol²³ with modifications. Briefly, approximately 4 million PBMCs per sample were fixed with 1% formaldehyde for 30 minutes at room temperature and quenched with 0.125M glycine for 5 minutes. Following cell and nuclei lysis, chromatin was sonicated using a Covaris S220 instrument. For the immunoprecipitation step chromatin was incubated overnight with 6 μl of H3K27ac antibody (Cell Signaling, #4353). Following immobilization on protein G beads and washing steps, samples were treated with proteinase K and crosslinks were reversed overnight at 65 C. The DNA was purified with Nucleomag beads and tested for enrichment with qPCR using specific primers. CHIP-sew libraries were prepared using the NEBNEXT® Ultra™ II DNA Library Prep Kit for Illumina. DNA concentration was measured with a Qubit fluorometer and size was assessed with the Agilent bioanalyzer DNA1000 kit. Samples were sequenced in a NovaSeq 6000 Illumina sequencer at the Greek Genome Center in BRFAA.

The study primary endpoint was the cytokine-production capacity of circulating PBMCs following stimulation with bacterial lipopolysaccharide (LPS). This was independently validated in the discovery cohort and both validation cohorts.

### Results

### Patients

Both validation cohorts were analyzed as follows: a) patients were grouped into phenotypes of PACS based on the 16-symptoms questionnaire.

These phenotypes were validated using the Symptom Tool-Impact Tool (ST-IT) questionnaire and the Short Form (SF)-36 health survey; b) we compared the distribution of phenotype groups of PACS between patients treated with placebo and patients treated with anakinra who participated in the SAVE-MORE trial; c) we analyzed the cytokine production capacity from circulating peripheral blood mononuclear cells (PBMCs) and serum biomarkers; comparisons were done with sex-ad age-matched healthy volunteer comparators; and d) we studied respiratory function through spirometry and carbon monoxide diffusing capacity (DLco) (FIG. 14. The demographics and comorbidities of patients and comparators were similar.

TABLE 2
Demographics of the discovery and of the validation cohorts.

| | | | | | | Validation cohorts | | | | | | |
|---|---|---|---|---|---|---|---|---|---|---|---|---|
| | Discovery cohort | | | | | Cohort 1 | | | | | | |
| | Patients | | Comparators | | | Patients | | Comparators | | | Cohort 2 | |
| | (n = 46) | | (n = 25) | | p | (n = 464) | | (n = 58) | | p | n = 130 | |
| Days from diagnosis, | 120 | (89-180) | NA | | — | 140 | (120-210) | NA | | — | 140 | (120-210) |
| median (Q1-Q3) | | | | | | | | | | | | |
| Severe COVID-19 | 12 | (26.1) | NA | | — | 367 | (79.1) | NA | | — | 95 | (73.1) |
| at acute | | | | | | | | | | | | |
| pneumonia, n (%) | | | | | | | | | | | | |
| Dexamethasone | 12 | (26.1) | NA | | — | 366 | (78.9) | NA | | — | 96 | (73.8) |
| treatment at acute | | | | | | | | | | | | |
| pneumonia, n (%) | | | | | | | | | | | | |
| CCI, mean ± SD | 1.2 | (1.7) | 1.6 | (2.2) | 0.371 | 2.2 | (1.6) | 1.7 | (0.4) | 0.099 | 2.6 | (1.7) |
| Age in years, | 49 | (16) | 16 | (64) | 0.543 | 60.8 | (12.1) | 60.3 | (4.9) | 0.841 | 49.5 | (14.0) |
| mean (SD) | | | | | | | | | | | | |
| Male sex, n (%) | 30 | (62.2) | 16 | (64.0) | 1.00 | 264 | (56.9) | 32 | (55.7) | 0.888 | 67 | (51.5) |
| Vaccination | | | | | | | | | | | | |
| status, n (%) | | | | | | | | | | | | |
| Nil dose | 100 | | 100 | | 1.00 | 25 | (5.4) | 0 | | | 26 | (20.0) |
| 2 doses | 0 | | 0 | | 0 | 439 | (94.6) | 58 | (100) | 0.096 | 68 | (52.3) |
| 3 doses | 0 | | 0 | | 0 | 0 | | 0 | | | 36 | (27.7) |
| Anti-SARS-CoV-2, U/I | | | | | | | | | | | | |
| median (range) | | | | | | | | | | | | |
| IgG | 72.6 | (0.4->2500) | 0.4 | (0.4-5.5) | <0.0001 | 1277.5 | (535->2500) | 10 | (0.6->2500) | 0.003 | 1756 | (13.6->2500) |
| IgM | 0.4 | (0.2-15.7) | 0.2 | (0.2-6.8) | <0.0001 | 0.3 | (0.2-0.8) | 0.2 | (0.2-0.3) | 0.119 | 0.3 | (0.2-1.4) |
| IgA | 2.3 | (0.1-9.9) | 0.2 | (0.1-5.3) | <0.0001 | 9.6 | (2.7-10.0) | 0.6 | (0.3-8.8) | 0.016 | 8.8 | (0.5-10) |

Abbreviations
COVID: Coronavirus disease;
CCI: Charlsons' comorbidity index;
NA: not applicable;
Q: quartile;
SD: standard deviation

Patients of the discovery cohort were not vaccinated against SARS-CoV-2 since vaccines were not available during enrolment in the present study. The concentrations of the anti-SARS-CoV-2 antibodies were significantly higher in the validation cohorts. All participants in the SAVE-MORE trial were hospitalized for radiologically confirmed pneumonia by SARS-CoV-2; had circulating concentrations of the biomarker suPAR (soluble urokinase plasminogen activator receptor) 6 ng/ml or more; and the majority had severe pneumonia according to the WHO definition necessitating oxygen supplementation and treatment with remdesivir and dexamethasone (Kyriazopoulou, et al, supra). Anakinra treatment was 100 mg once daily subcutaneously for 10 days. None of the patients was admitted in an Intensive Care Unit during enrolment. In the period of 120 days (median) after hospital discharge before enrolment in the validation cohort 1, most of these patients received two doses of anti-SARS-CoV-2 vaccine. Patients of the validation cohort 2 were treated during the acute infection with same SoC as SAVE-MORE participants (dexamethasone, remdesivir) but not anakinra.

### Primary Endpoint

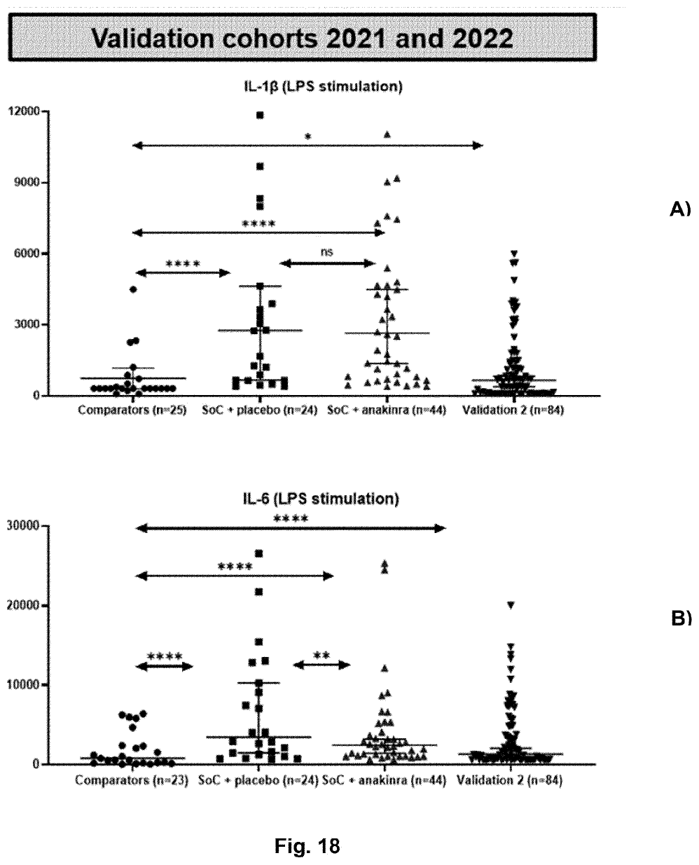

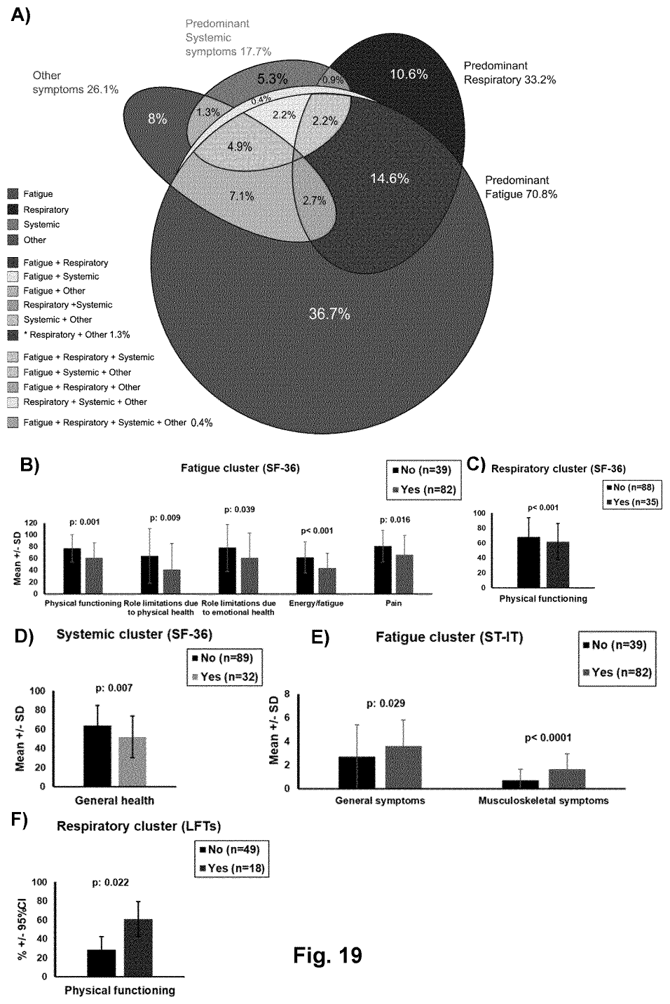

The primary endpoint of the study was the cytokine-production capacity of circulating PBMCs following stimulation with bacterial lipopolysaccharide (LPS). The production capacity for both IL-1β and IL-6 was significantly higher in patients recovering from COVID-19 pneumonia than comparators. This was independently validated in the discovery cohort (FIG. 2 a)-b)) and both validation cohorts (FIG. 18 a)-b)). Patients of the validation cohort 1 were divided into those who were treated with placebo and into those who were treated with anakinra. IL-6 production capacity was lower from the PBMCs of anakinra-treated patients. Notably, cytokine production capacity in the validation cohorts was greater than the discovery cohort. We believe that this difference is due to the vaccination status of the two cohorts; most patients in the validations cohorts were vaccinated against SARS-CoV-2 post hospital discharge whereas during the discovery cohort vaccines were not available. This is also reflected in the difference of antibody concentrations against SARS-CoV-2 between the discovery and the validation cohorts. Using receiver operator curve (ROC) analysis, it was found that production of IL-1β≥400 pg/ml and of IL-6≥625 pg/ml could distinguish patients from comparators. The odds for IL-1β production greater than 400 pg/ml among PACS patients was 4.81 (95% confidence intervals 1.97-11.74; p<0.0001). The respective odds for IL-6 production greater than 625 pg/ml was 11.75 (95% confidence intervals 4.23-32.67; p<0.0001).

### Post-Covid Symptoms

Among the 46 participants in the discovery cohort, 14 patients (30.4%) had signs compatible with PACS. The most common symptom was fatigue presenting in six patients (8.5%).

In order to identify specific phenotype clusters of PACS, we studied together patients of both validation cohorts 1 and 2. Subsequently, validation cohort 1 was studied to define the impact of IL-1 blockade during the acute phase of the disease for the progression to PACS, as these patients were treated with placebo or anakinra during their hospital admission for COVID-19.

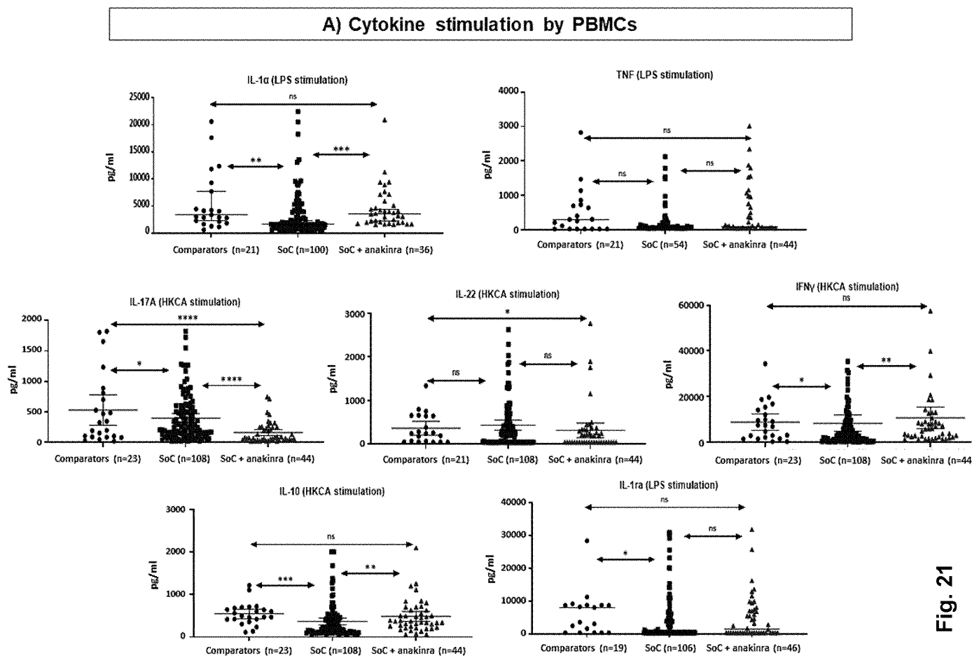

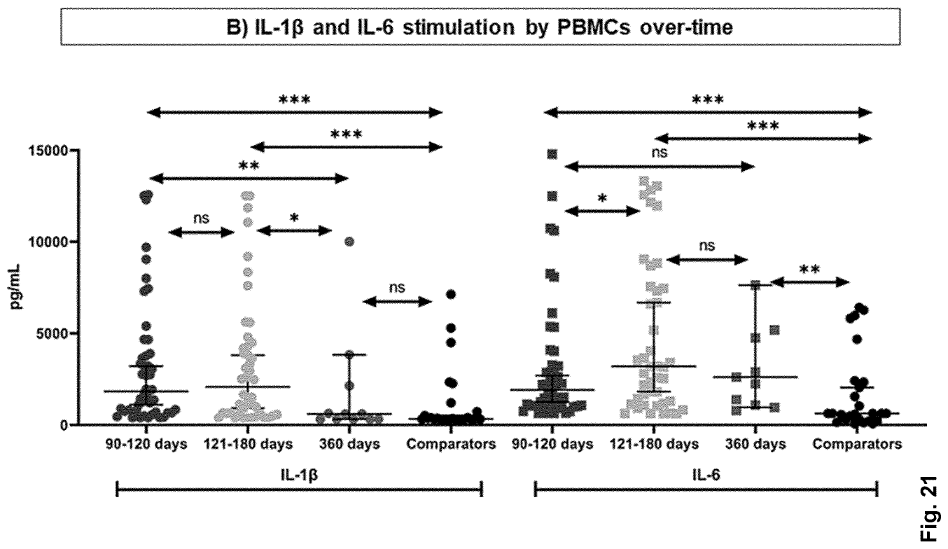

Using the 16-element questionnaire, we defined five clusters of patients: a) those who were asymptomatic and who responded negative to all questions; b) those with fatigue (70.8% among those with positive questionnaire); c) those with respiratory symptoms (33.2% among those with positive questionnaire); d) those with systemic symptoms (17.7% among those with positive questionnaire); and e) those with other symptoms (26.1% among those with positive questionnaire) (FIG. 19A). Using the answers provided to the SF-36 and the ST/IT questionnaires, we validated the adequacy of our used 16-element questionnaire to cluster PACS phenotypes (FIGS. 21B to 21F).

Based on this definition of clusters, patients of the validation cohort 1 could be asymptomatic or classified into one or more clusters of PACS. The worse PACS classification involves patients presenting with all four clusters. The odds for this worse PACS outcome for anakinra-treated patients was 0.59 (FIG. 13) which supports the hypothesis that progression into PACS is indeed driven at least partially by IL-1.

Patients of the validation cohort 1 were contacted by phone calls one year after COVID-19 infection and were asked for their health status. Finally, 271 patients of the 464 patients responded to the call, of which 85 patients were originally allocated to treatment with placebo and 186 patients were originally allocated to treatment with anakinra; the odds for progression into worse severity of PACS was 0.45 for anakinra treated patients (results not shown). The incidence of new disorders was similar between the two groups (results not shown). However, nine (10.6%) and seven (3.8%) of these patients were in need of new hospitalization which was not related to PACS (p: 0.048) (results not shown).

### Inflammatory Mediators in Plasma and Th1/Th2/Th17 Responses

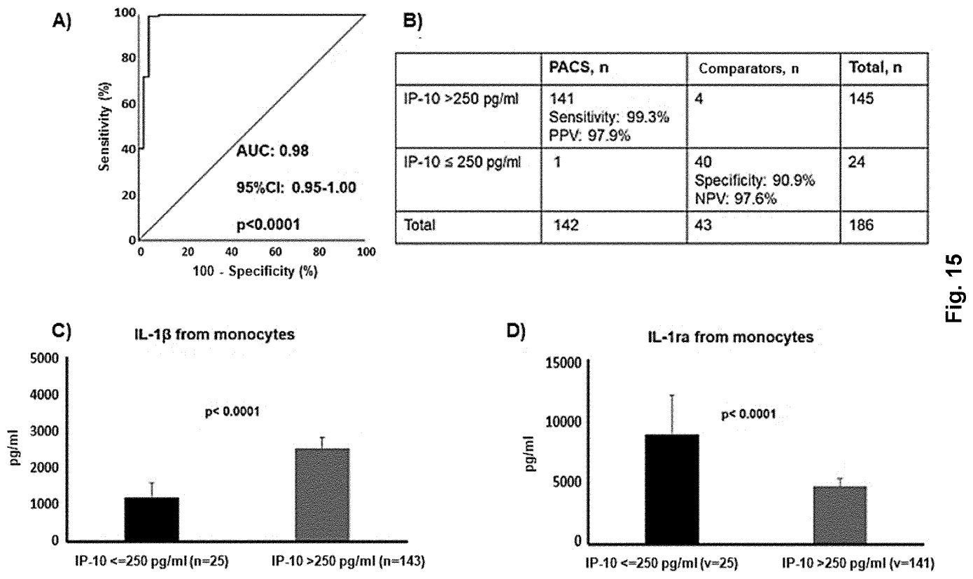

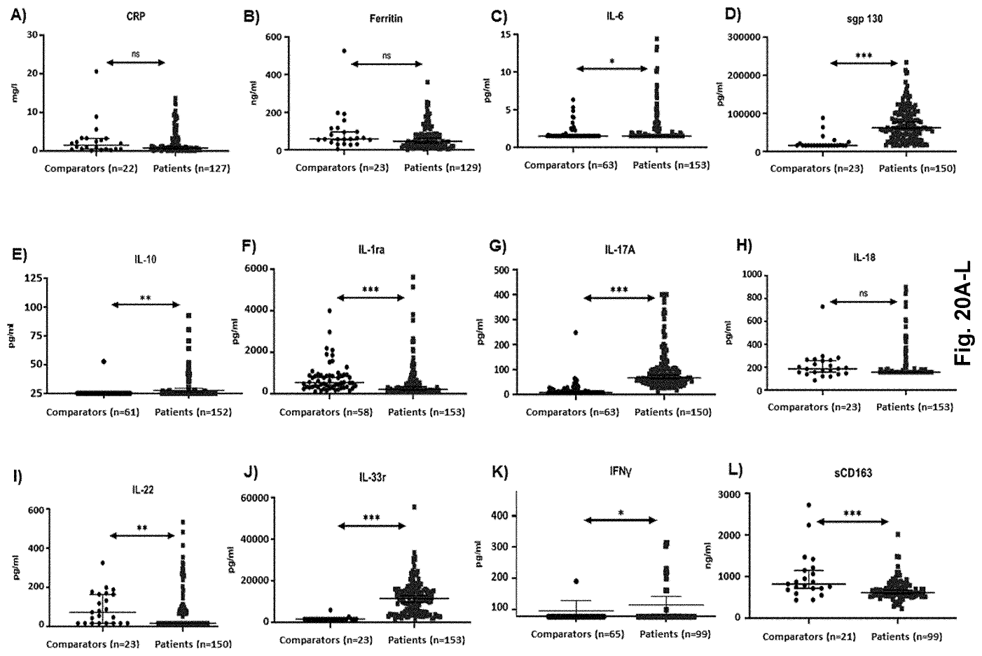

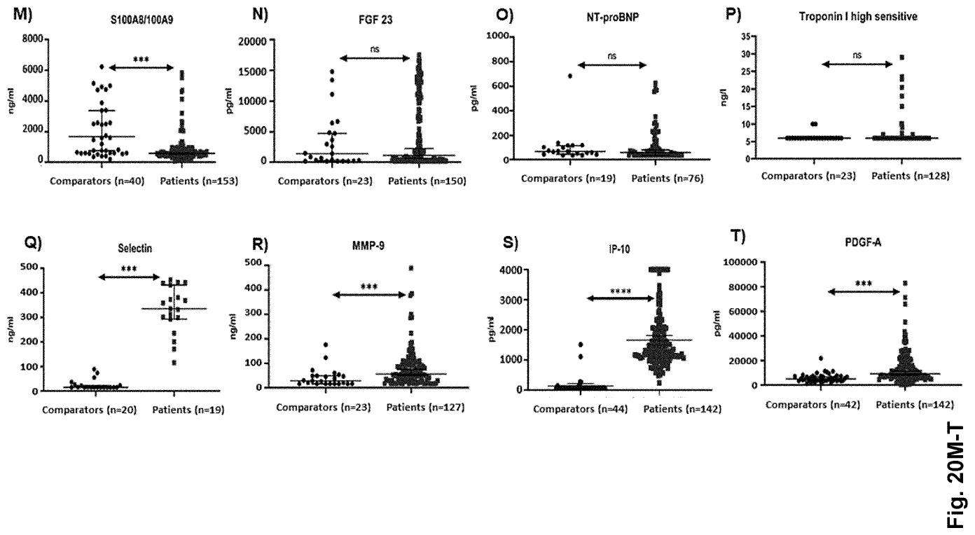

Several differences were found between comparators and patients post-COVID in circulating mediators. More precisely, among measured pro-inflammatory mediators IL-6, soluble glycoprotein (sgp)-130, IL-17A, IL-22, interferon (IFN)γ, s-Selectin, metalloproteinase (MMP)-9, IFNγ-induced protein (IP)-10, and platelet-derived growth factor (PDGF)-A were higher in post-COVID pneumonia than comparators; sCD163 was lower. Among the anti-inflammatory mediators, concentration of IL-10 and IL-33r was higher whereas IL-1ra concentration was lower than healthy comparators (FIG. 20). Using the Youden index of the Receiver Operator Characteristics curve, it was found that concentrations greater than 250 pg/ml have sensitivity 99.3%, specificity 90.9%, positive predictive value (PPV) 97.9% and negative predictive value (NPV) 97.6% for the differentiation between post-COVID state and comparators (FIGS. 15A & B).

The results of FIG. 18 showing similar cytokine production capacity among patients of the validation cohort 1 treated with placebo for the acute COVID-19 and of patients of the validation cohort 2 (no anakinra treatment during the acute infection) led to further analysis of cytokine production using both these patients together. TNF and IL-22 production was unaltered; the production of the anti-inflammatory IL-1ra and IL-10 decreased post-COVID-19; the production of the pro-inflammatory IL-17 A decreased as well (FIG. 21A). This long-term dysregulation of cytokine production capacity was less strong among SAVE-MORE patients treated with anakinra for the acute pneumonia episode. In eleven patients, blood sampling was repeated 12 months after acute COVID-19; IL-1β and IL-6 production by circulating monocytes was decreased compared to the production measured before 180 days after acute illness, but it remained higher than comparators (FIG. 21B).

Lymphocyte subsets were measured in the discovery cohort and no differences were found between comparators and patients (results not shown).

The linear distribution of cytokine production from PBMCs and of circulating biomarkers led us to hypothesize that there may be distinct immunotypes among patients with different clusters of PACS. Comparisons led us to identify the presence of two immunotypes: predominantly fatigue and predominantly respiratory. These immunotypes have distinct characteristics with respect to cytokine production capacity of PBMCs and the concentration of circulating mediators. In patients of the fatigue immunotype, immune responses are attenuated: PBMCs produce less of the pro-inflammatory TNF; the proinflammatory biomarker MMP-9 is also decreased. In patients of the immunotype of respiratory symptoms, PBMCs produce more pro-inflammatory IL-1β, IL-1α and IL-6, while circulating pro-inflammatory IL-17A and sCD163 are also increased (results not shown). A correlation analysis showed that: i) circulating IL-1ra concentration is negatively associated with the production of IL-17 following stimulation of PBMCs with heat-killed Candida albicans; ii) circulating IL-17A concentration is positively associated with the production of IL-1β, of IL-1α and IL-6 following stimulation of PBMCs with LPS; iii) circulating IL-33r concentration is correlated with the production of IL-1β, of IL-1α and IL-6 following stimulation of PBMCs with LPS and with the production of IFNγ and of IL-10 following stimulation of PBMCs with HKCA; and iv) circulating sgp130 concentration is negatively associated with the production of IL-1α following stimulation of PBMCs with LPS and with the production of IL-10 following stimulation of PBMCs with HKCA.

TABLE 3
Significant correlations between soluble cytokines and cytokines
produced after stimulation of PBMCs. Analysis includes only patients
with at least one cluster of post-acute COVID syndrome.

| Cytokines | | | | |
|---|---|---|---|---|
| produced | | | | |
| after PBMCs | Soluble cytokines | | | |
| stimulation | IL-1ra | IL-17A | IL-33r | sgp130 |
| IL-1β LPS | | rₛ: +0.469 | rₛ: +0.315 | |
| stimulation | | p < 0.0001 | p: 0.004 | |
| IL-1α LPS | | rₛ: +0.391 | rₛ: +0.311 | rₛ: −0.344 |
| stimulation | | p < 0.0001 | p: 0.004 | p: 0.002 |
| IL-6 LPS | | rₛ: +0.373 | | |
| stimulation | | p < 0.0001 | | |
| IFNγ HKCA | | | rₛ: +0.363 | |
| stimulation | | | p < 0.0001 | |
| IL-10 HKCA | | | rₛ: +0.479 | rₛ: −0.527 |
| stimulation | | | p < 0.0001 | p < 0.0001 |
| IL-17 HKCA | rₛ: −0.353 | | | |
| stimulation | p: 0.001 | | | |

Abbreviations:
IFN, interferon,
IL, interleukin,
PBMCs, peripheral blood mononuclear cells;
sgp, soluble glycoprotein

### Discussion

In the present study it was demonstrated that a significant dysregulation of the capacity of monocytes and lymphocytes to produce cytokines persists long time after hospital discharge of patients for COVID-19 pneumonia. This immune dysregulation lasts for up to 12 months and occurs irrespective of the presence or absence of clinical symptoms of PACS. Major characteristics of this immune dysregulation are the increased IL-1β and IL-6 production by monocytes, accompanied by attenuated Th2 responses and T17 responses. The discovery cohort and both validations cohorts were enrolled within different time periods showing that the described findings may be generalizable for all viral variants.

PACS occurs in a subset of the patients and is characterized by considerable heterogeneity of symptoms. Although symptoms vary, associations between the attenuation of pro-inflammatory responses and fatigue were identified, as well as between the increased IL-1β and IL-6 production by circulating monocytes and the respiratory cluster of symptoms. The findings have been validated in independent cohorts of patients which were compared with control volunteers without history of COVID-19 infection and who were matched for age, sex and comorbidities. The presence of dysregulated cytokine production capacity in all post-COVID individuals, independently of the presence or not of symptoms, may suggest that the presence of the higher cytokine production does not play a direct role in PACS. However, the counterargument may be that the patients who develop PACS have an increased sensitivity to develop symptoms in the presence of a high cytokine milieu. Therefore, to investigate the potential causality between inflammation and PACS, we assessed the impact of anti-IL-1 therapy during the acute phase of the infection on the long-term symptoms post-COVID-19 in the SAVE-MORE clinical trial (Kyriazopoulou et al, supra): patients originally allocated to SoC and anakinra treatment displayed significantly lower incidence of PACS compared with patients treated with SoC and placebo. The anakinra protection from PACS lasted as long as one year after recovery. These effects of anakinra on PACS suggest that over-production of IL-1β during the acute phase of the disease is likely to have an important impact on the development of PACS (Schultheiß et al, supra). The analysis of the 90-day outcome of the SAVE-MORE trial demonstrates that anakinra treatment decreased significantly persisting symptoms of ambulatory patients (Akinosoglou, K., et al, eClinicalMedicine 56, 2023). The analysis presented here suggests that this benefit is prolonged for at least one year after hospital discharge.

The role of IL-1 pathway dysregulation during the acute phase of COVID-19 for the progression from acute COVID-19 pneumonia to PACS is supported by three observations: a) patients treated with anakinra during the acute phase of COVID-19 had decreased likelihood for progression into PACS; b) the PBMCs of patients post-COVID display excess production of IL-1β, IL-1α and IL-6, and c) H3K27ac histone mark is changed in genes of the IL-1/IL-6 pathway. In the same patients, Th17 responses were less strongly inhibited. Two more features of the immune dysregulation of PACS need to be underlined: the first is the increased circulating levels of sCD163 which are notable in the phenotype cluster dominated by respiratory symptoms and which reflect increased activation of tissue macrophages (Kyriazopoulou, K. et al, BMC Med 2017:15, 172); the second is the decreased circulating IL-1ra concentration and of the lower monocyte production of IL-1ra which reflects a defect of the host to counterbalance for the excess production of both IL-1β and IL-1α.

As in Example 1, the findings that post-COVID-19 is characterized by increased cytokine production capacity leads to the question regarding the molecular mechanisms mediating these effects.

In conclusion, patients recovering from COVID-19 pneumonia in need of hospitalization during acute infection present long-term post-infection immune dysregulation characterized by hyper-production of IL-1β and IL-6 from monocytes, and defects in Th2 and T17 responses. Down-regulated responses are associated with fatigue, while hyper-production of IL-1β, IL-1α and IL-6 is associated with abnormal lung function. Epigenetic modifications accompany cytokine production dysregulation, and the persistently enhanced cytokine production could suggest that a state of inappropriately activated trained immunity after COVID19 could contribute to PACS (DiNardo, A. R. et al, supra). Importantly, anti-IL-1 therapy with anakinra during the acute phase of the disease significantly decreased the incidence of PACS, arguing for the importance of innate immune dysregulation for the long-term effects of COVID-19. These data argue also for the necessity of prospective clinical trials of anti-inflammatory immunotherapy (e.g. anakinra) in patients with PACS.

1.-25. (canceled)

26. A method of treatment of Post-Acute COVID Syndrome (PACS), comprising administering a dose of at least approximately 100 mg of anakinra to a subject in need thereof.

27. The method according to claim 26, wherein said dose is approximately 100 mg of anakinra.

28. The method according to claim 26, wherein said dose is a daily dose.

29. The method according to claim 26, wherein said dose is administered on the same time ±2 hours every day of administration.

30. The method according to claim 26, wherein said treatment is continued for at least 7 days, such as at least 14 days, such as at least 21 days, such as at least 28 days.

31. (canceled)

32. The method according to claim 30, wherein said dose is administered on day 1 to day 28 of a treatment period.

33. The method according to claim 26, wherein said subject receives one or more additional treatment periods of at least 7 days, at least 14 days, at least 21 days or at least 28 days.

34. The method according to claim 26, wherein anakinra is administered by subcutaneous injection, such as subcutaneous self-injection.

35. The method according to claim 26, wherein the subject has a history of COVID-19 infection in the last 60 days or more, such as in the last 90 days or more.

36. The method according to claim 26, wherein said subject developed PACS at least 60 to 90 days following COVID-19.

37. The method according to claim 26, wherein said subject has an IP-10 serum level of at least 250 pg/ml.

38. The method according to claim 26, wherein the treatment is of at least one of impaired lung function, impaired cardiac function and abnormal immune function.

39. The method according to claim 38, wherein the subject to be treated has lungs with reduced ability to transfer oxygen to blood/low diffusion capacity, an abnormal chest, and/or abnormal immune function.

40. The method according to claim 38, wherein the following are signs of impaired lung function: DLCOcor <76% and TLC and/or FVC lower than 80% of predicted.

41. The method according to claim 39, wherein the following are signs of an abnormal chest: at least a total radiology score in HRCT of more than 20 or walking of a distance less than 500 m in the 6-minute walk test.

42. The method according to claim 38, wherein the following is a sign of impaired cardiac function: a decrease of left ventricular ejection fraction (LVEF).

43. The method according to claim 38, wherein the following are signs of abnormal immune function: excessive IL-1β levels of more than 2500 pg/ml after stimulation of peripheral blood mononuclear cells (PBMCs) with lipopolysaccharide (LPS).

44.-48. (canceled)

49. The method according to claim 26, wherein said anakinra is a pharmaceutical composition of anakinra.

50. A diagnostic assay for identifying a subject that has PACS and/or is susceptible to developing PACS, comprising the steps of (i) measuring the levels of one or more cytokines selected from IL-1α, IL-1β, IL-6, TNFα and/or IP-10 in a sample isolated from the subject; and (ii) comparing said levels to those of a control sample, wherein a subject that has increased levels of said one or more cytokines compared to the control sample is identified as having PACS and/or being susceptible to developing PACS.

51.-54. (canceled)

55. The method according to claim 49, wherein said pharmaceutical composition is Kineret®, a formulation of anakinra at pH 6.5 suitable for injection, such as subcutaneous injection, containing sodium citrate, sodium chloride, disodium EDTA, polysorbate 80 and water.
